(19) United States
(12) Patent Application Publication
(10) Pub. No.: US 2023/0203549 A1
(43) Pub. Date: Jun. 29, 2023

(54) MICROBIALLY PRODUCED PALM OIL SUBSTITUTES

(71) Applicant: C16 Biosciences, Inc., New York, NY (US)

(72) Inventors: Harold M. MCNAMARA, New York, NY (US); Shara TICKU, New York, NY (US); David HELLER, New York, NY (US); Corentin MOEVUS, New York, NY (US); Vladimir YONG-GONZALEZ, New York, NY (US)

(21) Appl. No.: 17/885,280

(22) Filed: Aug. 10, 2022

Related U.S. Application Data
(63) Continuation of application No. PCT/US2021/017458, filed on Feb. 10, 2021.
(60) Provisional application No. 63/061,521, filed on Aug. 5, 2020.
(60) Provisional application No. 62/972,299, filed on Feb. 10, 2020.

(51) Int. Cl. C12P 7/6463; A23D 9/007; A23D 9/04; C12P 7/6472

(52) U.S. Cl. CPC C12P 7/6463; A23D 9/007; A23D 9/04; C12P 7/6472

(57) ABSTRACT

The disclosure relates to microbial lipid compositions produced by oleaginous microorganisms as alternatives to plant-derived palm oil. The microbial lipid compositions may have one or more characteristics of plant-derived palm oil. These compositions may be fractionable or otherwise capable of separation into different states. Further provided are products produced by or comprising the microbial lipids.

### CROSS-REFERENCE TO RELATED APPLICATIONS

This application claims the benefit of priority to U.S. Provisional Application No. 62/972,299, filed on Feb. 10, 2020, and to U.S. Provisional Application No. 63/061,521, filed on Aug. 5, 2020, the contents of each of which are herein incorporated by reference in their entireties.

### FIELD OF THE DISCLOSURE

The present disclosure relates to environmentally friendly and sustainable alternatives to plant-derived palm oil. The palm oil alternatives are produced by oleaginous microorganism and share one or more features with plant-derived palm oils. These alternatives may also be fractionated, treated, and/or derivatized based on their intended use.

### BACKGROUND

Palm oil is currently the most widely produced vegetable oil on the planet, as it finds uses in the manufacture of a large variety of products. It is widely used in food, as a biofuel precursor, and in soaps and cosmetics. The global demand for palm oil is approximately 57 million tons and is steadily increasing. However, the high demand for palm oil has resulted in environmentally detrimental practices related to the expansion of plantations devoted to palm oil-producing plants. Palm oil production is a leading contributor to tropical deforestation, resulting in habitat destruction, increased carbon dioxide emissions, and local smog clouds across South East Asia.

Thus, there is an urgent need for palm oil alternatives that do not rely upon utilization of oil palms and incur the associated negative environmental costs.

### BRIEF SUMMARY

In one aspect, the present disclosure provides a refined, bleached, and/or deodorized (RBD) microbial oil composition produced by an oleaginous yeast.

In one aspect, the present disclosure provides a refined, bleached, and/or deodorized (RBD) microbial oil composition produced by an oleaginous yeast, wherein the composition comprises ergosterol and does not comprise campesterol, β-sitosterol, or stigmasterol.

In one aspect, the present disclosure provides a refined and/or deodorized microbial oil composition produced by an oleaginous yeast, wherein the composition comprises at least one pigment selected from the group consisting of carotene, torulene and torulorhodin and does not comprise chlorophyll.

In some embodiments, the composition is bleached, thereby producing an RBD microbial oil composition, but wherein a measurable amount of the pigment remains.

In one aspect, the present disclosure provides a refined, bleached, and/or deodorized (RBD) microbial oil composition produced by an oleaginous yeast, wherein the composition is fractionable into two fractions, wherein the two fractions are microbial olein and microbial stearin, wherein each fraction comprises at least 10% of the composition's original mass, and wherein the iodine value (IV) of the fractions differs by at least 10.

In one aspect, the present disclosure provides a microbial oil composition produced by an oleaginous yeast, wherein the composition comprises the following amounts of fatty acids relative to the total fatty acids: at least about 30% w/w saturated fatty acids with chain lengths between 16 and 18 carbons long; at least about 30% w/w unsaturated fatty acids with 18 carbon chain lengths; and less than about 30% w/w total polyunsaturated fatty acids.

In one aspect, the present disclosure provides a refined, bleached, and/or deodorized (RBD) microbial oil composition produced by an oleaginous yeast, wherein the composition has one or more characteristics similar to plant-derived palm oil selected from the group consisting of: apparent density, refractive index, saponification value, unsaponifiable matter, iodine value, slip melting point, fatty acid composition, triglyceride content, overall saturation level, and level of mono- and poly-unsaturated fatty acids.

In one aspect, the present disclosure provides a microbial oil composition produced by an oleaginous yeast, comprising: at least about 30% w/w saturated fatty acids with chain lengths between 16 and 18 carbons long; at least about 30% w/w unsaturated fatty acids with 18 carbon chain lengths; less than about 30% w/w total polyunsaturated fatty acids; at least about 50 ppm ergosterol; wherein the composition does not contain a phytosterol or chlorophyll, and wherein the composition has one or more characteristics similar to plant-derived palm oil selected from the group consisting of iodine value, triglyceride content, slip melting point, oxidative stability, and overall saturation level.

In some embodiments, the composition comprises 10-45% C16 saturated fatty acid.

In some embodiments, the composition comprises 10-70% C18 unsaturated fatty acid.

In some embodiments, the composition comprises 3-30% C18 saturated fatty acid.

In some embodiments, the composition comprises a saponification value similar to that of plant-derived palm oil.

In some embodiments, the composition comprises a saponification value of 150-210.

In some embodiments, the composition comprises an iodine value similar to that of plant-derived palm oil.

In some embodiments, the composition comprises an iodine value of 50-65.

In some embodiments, the composition comprises a slip melting point similar to that of plant-derived palm oil.

In some embodiments, the composition comprises a slip melting point of 30° C.-40° C.

In some embodiments, the composition comprises a saturated fatty acid composition similar to that of plant-derived palm oil.

In some embodiments, the composition comprises a saturated fatty acid composition of at least 30%.

In some embodiments, the composition comprises a saturated fatty acid composition of at most 70%.

In some embodiments, the composition comprises an unsaturated fatty acid composition similar to that of plant-derived palm oil.

In some embodiments, the composition comprises an unsaturated fatty acid composition of at least 30%.

In some embodiments, the composition comprises an unsaturated fatty acid composition of at most 70%.

In some embodiments, the composition comprises a mono- and poly-unsaturated fatty acid composition similar to that of plant-derived palm oil.

In some embodiments, the composition comprises 30-50% mono-unsaturated fatty acids as a percentage of overall fatty acids.

In some embodiments, the composition comprises 5-25% poly-unsaturated fatty acids as a percentage of overall fatty acids.

In some embodiments, the composition comprises a triglyceride content similar to that of plant-derived palm oil.

In some embodiments, the composition comprises a triglyceride content of 90-98% as a percentage of overall glycerides.

In some embodiments, the composition comprises less than 100 ppm of, comprises less than 50 ppm of, or does not comprise a sterol selected from a phytosterol, cholesterol, or a protothecasterol.

In some embodiments, the composition comprises less than 100 ppm of, comprises less than 50 ppm of, or does not comprise a phytosterol.

In some embodiments, the composition comprises less than 100 ppm of, comprises less than 50 ppm of, or does not comprise a phytosterol selected from the group consisting of campesterol, β-sitosterol, stigmasterol.

In some embodiments, the composition comprises less than 100 ppm of, comprises less than 50 ppm of, or does not comprise cholesterol.

In some embodiments, the composition comprises less than 100 ppm of, comprises less than 50 ppm of, or does not comprise protothecasterol.

In some embodiments, the composition comprises ergosterol, comprises at least 50 ppm ergosterol, or comprises at least 100 ppm ergosterol.

In some embodiments, the composition comprises an ergosterol content of at least 60% w/w as a percentage of overall sterols.

In some embodiments, the composition does not comprise a pigment.

In some embodiments, the composition does not comprise chlorophyll.

In some embodiments, the composition comprises a pigment selected from the group consisting of carotene, torulene and torulorhodin.

In some embodiments, the composition comprises each of carotene, torulene and torulorhodin.

In some embodiments, the composition comprises at least 10 ppm, at least 50 ppm, or at least 100 ppm carotene.

In some embodiments, the composition comprises carotene, and wherein the carotene is β-carotene and/or a derivative thereof.

In some embodiments, the composition comprises at least 10 ppm, at least 50 ppm, or at least 100 ppm torulene and/or a derivative thereof.

In some embodiments, the composition comprises at least 10 ppm, at least 50 ppm, or at least 100 ppm torulorhodin and/or a derivative thereof.

In some embodiments, the oleaginous yeast is a recombinant yeast.

In some embodiments, the oleaginous yeast is of the genus Yarrowia, Candida, Rhodotorula, Rhodosporidium, Metschnikowia, Cryptococcus, Trichosporon, or Lipomyces.

In some embodiments, the oleaginous yeast is of the genus Rhodosporidium.

In some embodiments, the oleaginous yeast is of the species Rhodosporidium toruloides.

In some embodiments, the composition is fractionable.

In some embodiments, the composition may be fractionated into microbial olein and microbial stearin.

In some embodiments, the composition may be fractionated into microbial olein and microbial stearin, and wherein each fraction comprises at least 10% of the composition's starting mass.

In some embodiments, the composition may be fractionated into microbial olein and microbial stearin, and wherein the iodine value (IV) of the fractions differs by at least 10.

In some embodiments, the composition may be fractionated into microbial olein and microbial stearin, and wherein the IV of the fractions differs by at least 20.

In some embodiments, the composition may be fractionated into microbial olein and microbial stearin, and wherein the IV of the fractions differs by at least 30.

In one aspect, the present disclosure provides a microbial oil composition produced by an oleaginous yeast, wherein the composition comprises: less than 10% w/w palmitic-palmitic-palmitic triglycerides; greater than 15% w/w palmitic-palmitic-oleic triglycerides; and greater than 15% w/w oleic-oleic-palmitic triglycerides.

In some embodiments, said palmitic-palmitic-palmitic triglyceride content is between about 0.8% and 1.3% w/w.

In some embodiments, said palmitic-palmitic-oleic triglyceride content is between about 16.9% and 28.2% w/w.

In some embodiments, said oleic-oleic-palmitic triglyceride content is between about 15.7% and 26.0% w/w.

In some embodiments, the composition further comprises a stearic-stearic-oleic triglyceride content of less than 10% w/w and a stearic-oleic-oleic triglyceride content of less than 10% w/w.

In some embodiments, said stearic-stearic-oleic triglyceride content is between about 1.2% and 1.9% w/w.

In some embodiments, said stearic-oleic-oleic triglyceride content is between about 3.2% and 5.4% w/w.

In one aspect, the present disclosure provides a microbial oil composition produced by an oleaginous yeast, wherein the composition comprises triglycerides, and wherein greater than 40% of said triglycerides have one unsaturated sidechain.

In some embodiments, greater than 30% of said triglycerides have two unsaturated sidechains.

In some embodiments, between 10% and 15% of palmitic and/or stearic fatty acids are located at the sn-2 position of triglyceride molecules.

In one aspect, the present disclosure provides a microbial oil composition produced by an oleaginous yeast, wherein the composition comprises the following amounts of fatty acids relative to the total fatty acids: between about 7.0% and 35% stearic acid; between about 10% and 50% oleic acid; and between about 8% and 20% linoleic acid.

In one aspect, the present disclosure provides a method of producing a microbial oil composition according to any one of the foregoing embodiments, the method comprising the steps of: providing an oleaginous yeast and a carbon source, and culturing said oleaginous yeast, thereby producing said microbial oil.

In some embodiments, the methods and compositions recited in International Patent Application No. PCT/US2021/015302, incorporated by reference herein, are employed in the compositions and methods of the disclosure. In some embodiments, the feedstocks of International Patent Application No. PCT/US2021/015302 are utilized in the compositions and methods of the present disclosure.

### BRIEF DESCRIPTION OF THE DRAWINGS

The accompanying figures, which are incorporated herein and form a part of the specification, illustrate some, but not the only or exclusive, example embodiments and/or features. It is intended that the embodiments and figures disclosed herein are to be considered illustrative rather than limiting.

FIG. 1A shows a chromatogram of the fatty acid composition analysis of exemplary crude microbial oil; FIG. 1B shows a chromatogram of the fatty acid composition analysis of exemplary crude palm oil; FIG. 1C shows a chromatogram of the fatty acid composition analysis of exemplary crude hybrid palm oil; and FIG. 1D shows a bar graph of representative fatty acid compositions of microbial oil and palm oil.

FIG. 2A shows a chromatogram of the triglyceride composition analysis of exemplary crude microbial oil; FIG. 2B shows a chromatogram of the triglyceride composition analysis of exemplary crude palm oil; and FIG. 2C shows a chromatogram of the triglyceride composition analysis of exemplary crude hybrid palm oil.

FIG. 3 shows a chromatogram of the tocopherols analysis of exemplary crude microbial oil, crude palm oil, and crude hybrid palm oil. Notable peaks are annotated, with “External ISTD” illustrating the location of the standard.

FIG. 4A-4B show the results of a fatty acid analysis of exemplary microbial oils of the disclosure produced by three illustrative strains of the oleaginous yeast R. toruloides. FIG. 4A shows the overall fatty acid composition broken down by percentage of poly-unsaturated fatty acid (PUFA), mono-unsaturated fatty acid (MUFA), and saturated fatty acid (SFA). FIG. 4B shows the breakdown of the fatty acid composition for the microbial oils in terms of specific fatty acids.

FIG. 5A-5B show the results of fractionation on fatty acid composition for an exemplary microbial oil. FIG. 5A shows the results of fractionation on overall fatty acid composition in terms of PUFA, MUFA, and SFA. FIG. 5B shows the breakdown in terms of specific fatty acids for the crude microbial oil and each of the fractions.

FIG. 6A-6B show a visual comparison of fractionated microbial oils, non-fractionating microbial oil, and fractionated palm oil. FIG. 6A, left shows the visual results of fractionation on a microbial oil from R. toruloides; on the right is a fractionated palm oil. FIG. 6B shows the visual results of fractionation on a fractionable microbial oil (left) and a non-fractionating microbial oil (right).

FIG. 7A-7D show total ion chromatograms for four different oil samples: an exemplary R. toruloides microbial oil of the disclosure (FIG. 7A); algae oil (FIG. 7B); crude palm oil (FIG. 7C); and refined, bleached, and deodorized (RBD) palm oil (FIG. 7D).

FIG. 8 shows a representative extracted peak for a compound of interest (ergosterol-TMS) from the total ion chromatogram of an exemplary microbial oil of the present disclosure.

FIG. 9A-9E show the electron-ionization spectra for five different derivatized sterols spiked into crude palm oil: ergosterol-TMS (FIG. 9A); cholesterol-TMS (FIG. 9A); campesterol-TMS (FIG. 9A); sitosterol-TMS (FIG. 9A); and stigmasterol-TMS (FIG. 9A).

FIG. 10A-10B show the results of a carotenoid analysis of agricultural palm oil. FIG. 10A shows the overall UV/Vis absorbance spectrum. FIG. 10B shows the HPLC-DAD chromatogram with absorbance at 450 nm.

FIG. 11A-11B show the results of a carotenoid analysis of a strong acid-extracted exemplary R. toruloides microbial oil of the present disclosure. FIG. 11A shows the overall UV/Vis absorbance spectrum. FIG. 11B shows the HPLC-DAD chromatogram with absorbance at 450 nm.

FIG. 12A-12B show the results of a carotenoid analysis of a strong acid-extracted exemplary R. toruloides microbial oil of the present disclosure. FIG. 12A shows the overall UV/Vis absorbance spectrum. FIG. 12B shows the HPLC-DAD chromatogram with absorbance at 450 nm.

FIG. 13A-13B show the results of a carotenoid analysis of a weak acid-extracted exemplary R. toruloides microbial oil of the present disclosure. FIG. 13A shows the overall UV/Vis absorbance spectrum. FIG. 13B shows the HPLC-DAD chromatogram with absorbance at 450 nm.

FIG. 14A-14B show the results of a carotenoid analysis of an acid-free extracted exemplary R. toruloides microbial oil of the present disclosure. FIG. 14A shows the overall UV/Vis absorbance spectrum. FIG. 14B shows the HPLC-DAD chromatogram with absorbance at 450 nm.

FIG. 15A-15B show the results of a carotenoid analysis of an acid-free extracted exemplary R. toruloides microbial oil of the present disclosure. FIG. 15A shows the overall UV/Vis absorbance spectrum. FIG. 15B shows the HPLC-DAD chromatogram with absorbance at 450 nm.

### DETAILED DESCRIPTION

The following description includes information that may be useful in understanding the present disclosure. It is not an admission that any of the information provided herein is prior art or relevant to the presently claimed disclosures, or that any publication specifically or implicitly referenced is prior art.

### Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art.

As used herein, the singular forms “a,” “an,” and “the: include plural referents unless the content clearly dictates otherwise.

The term “about” or “approximately” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

A “fatty acid” is a carboxylic acid with a long aliphatic chain, which is either saturated or unsaturated. Most naturally occurring fatty acids have an unbranched chain of an even number of carbon atoms, from 4 to 28. Fatty acids are usually not found free in organisms, but instead within three main classes of esters: triglycerides, phospholipids, and cholesteryl esters. Within the context of this disclosure, a reference to a fatty acid may refer to either its free or ester form.

“Fatty acid profile” as used herein refers to how specific fatty acids contribute to the chemical composition of an oil.

As used herein, the term “fractionable” is used to refer to a microbial oil or lipid composition which can be separated into at least two fractions that differ in saturation levels and wherein the at least two fractions each make up at least 10% w/w (or mass/mass) of the original microbial oil or lipid composition. In some embodiments, the saturation levels of the fractions are characterized by their iodine value (IV). In some embodiments, the IV of the fractions differs by at least 10. Accordingly, a “fraction” as used herein refers to a separable component of a microbial oil that differs in saturation level from at least one other separable component of the microbial oil.

“Lipid” means any of a class of molecules that are soluble in nonpolar solvents (such as ether and hexane) and relatively or completely insoluble in water. Lipid molecules have these properties, because they are largely composed of long hydrocarbon tails that are hydrophobic in nature. Examples of lipids include fatty acids (saturated and unsaturated); glycerides or glycerolipids (such as monoglycerides, diglycerides, triglycerides or neutral fats, and phosphoglycerides or glycerophospholipids); and nonglycerides (sphingolipids, tocopherols, tocotrienols, sterol lipids including cholesterol and steroid hormones, prenol lipids including terpenoids, fatty alcohols, waxes, and polyketides).

“Microorganism” and “microbe” mean any microscopic unicellular organism and can include bacteria, algae, yeast, or fungi.

“Oleaginous” as used herein refers to material, e.g., a microorganism, which contains a significant component of oils, or which is itself substantial composed of oil. An oleaginous microorganism can be one that is naturally occurring or synthetically engineered to generate a significant proportion of oil.

“Oleaginous yeast” as used herein refers to a collection of yeast species that can accumulate a high proportion of their biomass as lipids (namely greater than 20% of dry cell mass). An oleaginous yeast can be one that is naturally occurring or synthetically engineered to generate a significant proportion of oil.

As used herein, “RBD” refers to refinement, bleaching, and deodorizing or refers to an oil that has undergone these processes.

“Rhodosporidium toruloides” refers to a particular species of oleaginous yeast. Previously called Rhodotorula glutinis or Rhodotorula gracilis. Also abbreviated as R. toruloides. This species includes multiple strains with minor genetic variation.

For the purposes of this disclosure, “single cell oils,” “microbial oils,” “lipid composition” and “oils” refer to microbial lipids produced by oleaginous microorganisms.

“Tailored fatty acid profile” as used herein refers to a fatty acid profile in a microbial oil which has been manipulated towards target properties, either by changing culture conditions, the species of oleaginous microorganism producing the microbial oil, or by genetically modifying the oleaginous microorganism.

“Triglyceride(s)” as used herein refers to a glycerol bound to three fatty acid molecules. They may be saturated or unsaturated, and various denominations may include other isomers. For example, reference to palmitic-oleic-palmitic (P-O-P) would also include the isomers P-P-O and O-P-P.

“W/W” or “w/w”, in reference to proportions by weight, refers to the ratio of the weight of one substance in a composition to the weight of the composition. For example, reference to a composition that comprises 5% w/w oleaginous yeast biomass means that 5% of the composition's weight is composed of oleaginous yeast biomass (e.g., such a composition having a weight of 100 mg would contain 5 mg of oleaginous yeast biomass) and the remainder of the weight of the composition (e.g., 95 mg in the example) is composed of other ingredients.

### Overview

The present disclosure relates to novel microbial lipids that have been refined, bleached, and/or deodorized. These lipids may serve as palm oil alternatives and be fractionated and/or used in a variety of downstream products of interest.

### Oleaginous Microorganisms

The present disclosure provides microbial lipids produced by oleaginous microorganisms. In some embodiments, the oleaginous microorganism is a microalgae, yeast, mold, or bacterium.

The use of oleaginous microorganisms for lipid production has many advantages over traditional oil harvesting methods, e.g., palm oil harvesting from palm plants. For example, microbial fermentation (1) does not compete with food production in terms of land utilization; (2) can be carried out in conventional microbial bioreactors; (3) has rapid growth rates; (4) is unaffected or minimally affected by space, light, or climate variations; (5) can utilize waste products as feedstock; (6) is readily scalable; and (7) is amenable to bioengineering for the enrichment of desired fatty acids or oil compositions. In some embodiments, the present methods have one or more of the aforementioned advantages over plant-based oil harvesting methods.

In some embodiments, the oleaginous microorganism is an oleaginous microalgae. In some embodiments, the microalgae is of the genus Botryococcus, Cylindrotheca, Nitzschia, or Schizochytrium. In some embodiments, the oleaginous microorganism is an oleaginous bacterium. In some embodiments, the bacterium is of the genus Arthrobacter, Acinetobacter, Rhodococcus, or Bacillus. In some embodiments, the bacterium is of the species Acinetobacter calcoaceticus, Rhodococcus opacus, or Bacillus alcalophilus. In some embodiments, the oleaginous microorganism is an oleaginous fungus. In some embodiments, the fungus is of the genus Aspergillus, Mortierella, or Humicola. In some embodiments, the fungus is of the species Aspergillus oryzae, Mortierella isabellina, Humicola lanuginosa, or Mortierella vinacea.

Oleaginous yeast in particular are robust, viable over multiple generations, and versatile in nutrient utilization. They also have the potential to accumulate intracellular lipid content up to greater than 70% of their dry biomass. In some embodiments, the oleaginous microorganism is an oleaginous yeast. In some embodiments, the yeast may be in haploid or diploid forms. The yeasts may be capable of undergoing fermentation under anaerobic conditions, aerobic conditions, or both anaerobic and aerobic conditions. A variety of species of oleaginous yeast that produce suitable oils and/or lipids can be used to produce microbial lipids in accordance with the present disclosure. In some embodiments, the oleaginous yeast naturally produces high (20%, 25%, 50% or 75% of dry cell weight or higher) levels of suitable oils and/or lipids. Considerations affecting the selection of yeast for use in the invention include, in addition to production of suitable oils or lipids for production of food products: (1) high lipid content as a percentage of cell weight; (2) ease of growth; (3) ease of propagation; (4) ease of biomass processing; and (5) glycerolipid profile. In some embodiments, the oleaginous yeast comprise cells that are capable of producing at least 20%, 25%, 50% or 75% or more lipid by dry weight. In other embodiments, the oleaginous yeast contains at least 25-35% or more lipid by dry weight.

Suitable species of oleaginous yeast for producing the microbial lipids of the present disclosure include, but are not limited to Candida apicola, Candida sp., Cryptococcus albidus. Cryptococcus curvatus, Cryptococcus terricolus, Cutaneotrichosporon oleaginosus, Debaromyces hansenii, Endomycopsis vernalis, Geotrichum carabidarum, Geotrichum cucujoidarum, Geotrichum histeridarum, Geotrichum silvicola, Geotrichum vulgare, Hyphopichia burtonii, Lipomyces hpofer, Lypomyces orentalis, Lipomyces starkeyi, Lipomyces tetrasporous, Pichia mexicana, Rodosporidium sphaerocarpum, Rhodosporidium toruloides Rhodotorula aurantiaca, Rhodotorula dairenensis, Rhodotorula diffluens, Rhodotorula glutinus, Rhodotorula glutinis var. glutinis, Rhodotorula gracilis, Rhodotorula graminis Rhodotorula minuta, Rhodotorula mucilaginosa, Rhodotorula mucilaginosa, Rhodotorula terpenoidahs, Rhodotorula toruloides, Sporobolomyces alborubescens, Starmerella bombicola, Torulaspora delbruekii, Torulaspora pretoriensis, Trichosporon behrend, Trichosporon brassicae, Trichosporon domesticum, Trichosporon laibachii, Trichosporon loubieri, Trichosporon loubieri, Trichosporon montevideense, Trichosporon pullulans, Trichosporon sp., Wickerhamomyces canadensis, Yarrowia hpolytica, and Zygoascus meyerae.

In some embodiments, the yeast is of the genera Yarrowia, Candida, Rhodotorula, Rhodosporidium, Metschnikowia, Cryptococcus, Trichosporon, or Lipomyces. In some embodiments, the yeast is of the genus Yarrowia. In some embodiments, the yeast is of the species Yarrowia lipolytica. In some embodiments, the yeast is of the genus Candida. In some embodiments, the yeast is of the species Candida curvata. In some embodiments, the yeast is of the genus Cryptococcus. In some embodiments, the yeast is of the species Cryptococcus albidus. In some embodiments, the yeast is of the genus Lipomyces. In some embodiments, the yeast is of the species Lipomyces starkeyi. In some embodiments, the yeast is of the genus Rhodotorula. In some embodiments, the yeast is of the species Rhodotorula glutinis. In some embodiments, the yeast is of the genus Metschnikowia. In some embodiments, the yeast is of the species Metschnikowia pulcherrima.

In some embodiments, the oleaginous yeast is of the genus Rhodosporidium. In some embodiments, the yeast is of the species Rhodosporidium toruloides. In some embodiments, the oleaginous yeast is of the genus Lipomyces. In some embodiments, the oleaginous yeast is of the species Lipomyces Starkeyi.

In some embodiments, the oleaginous microorganisms that produce the microbial lipids of the present disclosure are a homogeneous population comprising microorganisms of the same species and strain. In some embodiments, the oleaginous microorganisms that produce the microbial lipids of the present disclosure are a heterogeneous population comprising microorganisms from more than one strain. In some embodiments, the oleaginous microorganisms that produce the microbial lipids of the present disclosure are a heterogeneous population comprising two or more distinct populations of microorganisms of different species.

The oleaginous microorganisms that produce the microbial lipids of the present disclosure may have been improved in terms of one or more aspects of lipid production. These aspects may include lipid yield, lipid titer, dry cell weight titer, lipid content, and lipid composition. In some embodiments, lipid production may have been improved by genetic or metabolic engineering to adapt the microorganism for optimal growth on the feedstock. In some embodiments, lipid production may have been improved by varying one or more parameters of the growing conditions, such as temperature, shaking speed, growth time, etc. The oleaginous microorganisms of the present disclosure, in some embodiments, are grown from isolates obtained from nature (e.g., wild-types). In some embodiments, wild-type strains are subjected to natural selection to enhance desired traits (e.g., tolerance of certain environmental conditions such as temperature, inhibitor concentration, pH, oxygen concentration, nitrogen concentration, etc.). For example, a wild-type strain (e.g., yeast) may be selected for its ability to grow and/or ferment in a feedstock of the present disclosure, e.g., a feedstock comprising one or more microorganism inhibitors. In other embodiments, wild-type strains are subjected to directed evolution to enhance desired traits (e.g., lipid production, inhibitor tolerance, growth rate, etc.). In some embodiments, the cultures of microorganisms are obtained from culture collections exhibiting desired traits. In some embodiments, strains selected from culture collections are further subjected to directed evolution and/or natural selection in the laboratory. In some embodiments, oleaginous microorganisms are subjected to directed evolution and selection for a specific property (e.g., lipid production and/or inhibitor tolerance). In some embodiments, the oleaginous microorganism is selected for its ability to thrive on a feedstock of the present disclosure.

In some embodiments, directed evolution of the oleaginous microorganisms generally involves three steps. The first step is diversification, wherein the population of organisms is diversified by increasing the rate of random mutation creating a large library of gene variants. Mutagenesis can be accomplished by methods known in the art (e.g., chemical, ultraviolet light, etc.). The second step is selection, wherein the library is tested for the presence of mutants (variants) possessing the desired property using a screening method. Screens enable identification and isolation of high-performing mutants. The third step is amplification, wherein the variants identified in the screen are replicated. These three steps constitute a “round” of directed evolution. In some embodiments, the microorganisms of the present disclosure are subjected to a single round of directed evolution. In other embodiments, the microorganisms of the present disclosure are subjected to multiple rounds of directed evolution. In various embodiments, the microorganisms of the present disclosure are subjected to 2, 3, 4, 5, 6, 7, 8, 9, 10, 20, 30, 40, 50, 60, 70, 80, 90, or 100 or more rounds of directed evolution. In each round, the organisms expressing the highest level of the desired trait of the previous round are diversified in the next round to create a new library. This process may be repeated until the desired trait is expressed at the desired level.

### Properties of Microbial Oil

The present disclosure provides microbial oils produced by oleaginous microorganisms. In some embodiments, the microbial oils of the present disclosure are characterized by fatty acid composition, triglyceride composition, sterol composition, pigment composition, ability to be fractionated, slip melting point, iodine value, saponification value, and the like.

Sterol Composition

In some embodiments, the microbial oil comprises one or more sterols. In some embodiments, the microbial oil comprises ergosterol. In some embodiments, the microbial oil comprises at least 50, 100, 150, 200, 250, 300, 350, 400, 450, 500, 550, 600, 650, 700, 750, 800, 850, 900, 950, 1000, 1500, or 2000 ppm, or any ranges or subranges therebetween, of ergosterol. In some embodiments, the microbial oil comprises at least 50 ppm ergosterol. In some embodiments, the microbial oil comprises at least 100 ppm ergosterol. In some embodiments, at least 25, 30, 35, 40, 45, 50, 55, 60, 65, 70, 75, 80, 85, 90, or 95%, or any ranges or subranges therebetween, of the sterols in the microbial oil are ergosterol. In some embodiments at least 60% of the overall sterol composition is ergosterol.

In some embodiments, the microbial oil comprises less than 100 ppm of a phytosterol, cholesterol, or a protothecasterol. In some embodiments, the microbial oil comprises less than 50 ppm of of a phytosterol, cholesterol, or a protothecasterol. In some embodiments, the microbial oil does not comprise a sterol selected from a phytosterol, cholesterol, or a protothecasterol.

In some embodiments, the microbial oil does not comprise plant sterols. In some embodiments, the microbial oil does not comprise one or more phytosterols. In some embodiments, the microbial oil does not comprise campesterol, β-sitosterol, or stigmasterol. In some embodiments, the microbial oil does not comprise cholesterol. In some embodiments, the microbial oil does not comprise protothecasterol.

In some embodiments, the microbial oil comprises one or more sterols or stanols in addition to ergosterol. In some embodiments, the microbial oil comprises at least 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 300, 400, 500, 600, 700, 800, 900, or 1000 ppm, or any ranges or subranges therebetween, of one or more of 3,5-Cycloergosta-6,8(14),22-triene, anthraergostatetraenol p-chlorobenzoate, ergosta-5,7,9(11),22-tetraen-3β-ol, ergosta-7,22-dien-3-ol, 1′-Methyl-1′H-5α-cholest-3-eno[3,4-b]indole, 5χ-ergost-7-en-3β-ol, anthraergostatetraenol hexahydrobenzoate, 4,4-dimethylcholesta-8,24-dien-3-ol, and 9,19-cyclolanost-24-en-3-ol.

Pigments

In some embodiments, the microbial oil comprises a pigment. In some embodiments, the microbial oil comprises at least one pigment selected from the group consisting of carotene, torulene and torulorhodin.

In some embodiments, the microbial oil comprises carotene. In some embodiments, the microbial oil comprises at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 ppm, or any ranges or subranges therebetween, of carotene. In some embodiments, the microbial oil comprises at least 25 ppm of carotene. In some embodiments, the microbial oil comprises at least 50 ppm of carotene. In some embodiments, the microbial oil comprises at least 100 ppm of carotene. In some embodiments, the carotene is β-carotene and/or a derivative thereof. In some embodiments, the carotene is (13Z)-β-Carotene. In some embodiments, the carotene is (9Z)-β-Carotene.

In some embodiments, the microbial oil comprises torulene. In some embodiments, the microbial oil comprises torulorhodin. In some embodiments, the microbial oil comprises a derivative of torulene and/or torulorhodin. In some embodiments, the microbial oil comprises at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 ppm, or any ranges or subranges therebetween, of torulene, torulorhodin, and/or derivatives thereof. In some embodiments, the microbial oil comprises at least 25 ppm of torulene, torulorhodin, and/or derivatives thereof. In some embodiments, the microbial oil comprises at least 50 ppm of torulene, torulorhodin, and/or derivatives thereof. In some embodiments, the microbial oil comprises at least 100 ppm of torulene, torulorhodin, and/or derivatives thereof. In some embodiments, the microbial oil comprises at least 300 ppm of torulene, torulorhodin, and/or derivatives thereof.

In some embodiments, the microbial oil comprises each of carotene, torulene and torulorhodin. In some embodiments, the microbial oil does not comprise chlorophyll.

Fractionable

In some embodiments, the microbial oil is fractionable. In some embodiments, the microbial oil is fractionable into two or more fractions. In some embodiments, the microbial oil is fractionable into more than two fractions. In some embodiments, the microbial oil is fractionable into two fractions, which may then be further fractionated.

In some embodiments, the microbial oil is fractionable into two fractions. In some embodiments, the two fractions are microbial olein and microbial stearin. In some embodiments, each fraction comprises at least 10% of the microbial oil's original mass. In some embodiments, the iodine value (IV) of the fractions differs by at least 10. In some embodiments, the iodine value of the fractions differs by at least 20. In some embodiments, the iodine value of the fractions differs by at least 30.

Fatty Acid Composition

The composition of the microbial oil may vary depending on the strain of microorganism, feedstock composition, and growing conditions. In some embodiments, the microbial oil produced by the oleaginous microorganisms of the present disclosure comprise about 90% w/w triacylglycerol with a percentage of saturated fatty acids (% SFA) of about 44%. The most common fatty acids produced by oleaginous microbial fermentation on the present feedstocks are oleic acid (C18:1), stearic acid (C18:0), palmitic acid (C16:0), palmitoleic acid (C16:1), and myristic acid (C14:0).

In some embodiments, the microbial oil comprises myristic acid (C14:0). In some embodiments, the microbial oil comprises at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, or at least 5% myristic acid, or any ranges or subranges therebetween.

In some embodiments, the microbial oil comprises at least 5%, at least 10%, at least 15%, at least 20%, at least 25%, at least 30%, at least 35%, at least 40%, at least 45%, at least 50%, at least 55%, or at least 60% w/w palmitic acid (C16:0), or any ranges or subranges therebetween. In some embodiments, the microbial oil comprises at least 5% w/w palmitic acid. In some embodiments, the microbial oil comprises at least 10% w/w palmitic acid. In some embodiments the microbial oil comprises about 10-40% w/w palmitic acid. In some embodiments the microbial oil comprises about 13-35% w/w palmitic acid.

In some embodiments, the microbial oil comprises at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least 7%, at least 8%, at least 9% or at least 10% w/w palmitoleic acid (C16:1), or any ranges or subranges therebetween. In some embodiments, the microbial oil comprises at least 0.1% w/w palmitoleic acid. In some embodiments, the microbial oil comprises at least 0.5% w/w palmitoleic acid. In some embodiments, the microbial oil comprises about 0.5-10% w/w palmitoleic acid. In some embodiments, the microbial oil comprises about 0.5-5% w/w palmitoleic acid.

In some embodiments, the microbial oil comprises margaric acid (C17:0). In some embodiments, the microbial oil comprises at least 1%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, or at least 25% margaric acid, or any ranges or subranges therebetween. In some embodiments, the microbial oil comprises about 5-25% w/w margaric acid. In some embodiments, the microbial oil comprises about 9-21% w/w margaric acid.

In some embodiments, the microbial oil comprises at least 1%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, or at least 25% w/w stearic acid (C18:0), or any ranges or subranges therebetween. In some embodiments, the microbial oil comprises at least 1% w/w stearic acid. In some embodiments, the microbial oil comprises at least 5% w/w stearic acid. In some embodiments, the microbial oil comprises about 5-25% w/w stearic acid. In some embodiments, the microbial oil comprises about 9-21% w/w stearic acid.

In some embodiments, the microbial oil comprises at least 5%, at least 10%, at least 15%, at least 20%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54% at least 55%, at least 56%, at least 57%, at least 58%, at least 59%, or at least 60% w/w oleic acid (C18:1), or any ranges or subranges therebetween. In some embodiments, the microbial oil comprises at least 25% w/w oleic acid. In some embodiments, the microbial oil comprises at least 30% w/w oleic acid. In some embodiments, the microbial oil comprises about 30-65% w/w oleic acid. In some embodiments, the microbial oil comprises about 39-55% w/w oleic acid.

In some embodiments, the microbial oil comprises C18:2 (linoleic acid). In some embodiments, the microbial oil comprises at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, or at least 5% linoleic acid, or any ranges or subranges therebetween. In some embodiments, the microbial oil comprises about 5-25% linoleic acid. In some embodiments, the microbial oil comprises about 10-20% linoleic acid.

In some embodiments, the microbial oil comprises C18:3 (linolenic acid). In some embodiments, the microbial oil comprises at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, or at least 5% linolenic acid, or any ranges or subranges therebetween.

In some embodiments, the microbial oil comprises C20:0 (arachidic acid). In some embodiments, the microbial oil comprises at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, or at least 5% arachidic acid, or any ranges or subranges therebetween.

In some embodiments, the microbial oil comprises C24:0 (lignoceric acid). In some embodiments, the microbial oil comprises at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, or at least 5% lignoceric acid, or any ranges or subranges therebetween.

In some embodiments, the microbial oil comprises C12:0. In some embodiments, the microbial oil comprises C15:1. In some embodiments, the microbial oil comprises C16:1. In some embodiments, the microbial oil comprises C17:1. In some embodiments, the microbial oil comprises C18:3. In some embodiments, the microbial oil comprises C20:1. In some embodiments, the microbial oil comprises C22:0. In some embodiments, the microbial oil comprises C22:1. In some embodiments, the microbial oil comprises C22:2. In some embodiments, the microbial oil comprises about 0.1%, about 0.2%, about 0.3%, about 0.4%, about 0.5%, about 0.6%, about 0.7%, about 0.8%, about 0.9%, about 1%, about 2%, about 3%, about 4%, or about 5% of any one of these fatty acids, or any ranges or subranges therebetween. In some embodiments, the microbial oil comprises about 0-5% of any one of these fatty acids. In some embodiments, the microbial oil comprises about 0.1-2% of any one of these fatty acids.

Characteristics Similar to Plant-Derived Palm Oil

In some embodiments, the microbial oils of the present disclosure have differences from plant-derived palm oil. In some embodiments, these differences are useful and allow for manipulation of the microbial oil for the improved production of a given product compared to plant-derived palm oil. For example, in some embodiments, the fatty acid profile of a microbial oil is tailored so as to produce a higher fraction of one or more fatty acids of interest for use in production of a product. In some embodiments, other parameters of the microbial oil are also able to be manipulated for increased production of a component of interest or decreased production of an undesired component relative to plant-derived palm oil.

However, in some embodiments, the present compositions are also useful as environmentally friendly alternatives to plant-derived palm oil. Therefore, in some embodiments, the microbial oil has one or more properties similar to those of plant-derived palm oil. Exemplary properties include apparent density, refractive index, saponification value, unsaponifiable matter, iodine value, slip melting point, and fatty acid composition.

In some embodiments, the microbial oil has a fatty acid profile similar to that of plant-derived palm oil. In some embodiments, the microbial oil has a significant fraction of C16:0 fatty acid. In some embodiments, the microbial oil has a significant fraction of C18:1 fatty acid. In some embodiments, the microbial oil comprises 10-45% C16 saturated fatty acid. In some embodiments, the microbial oil comprises 10-70% C18 unsaturated fatty acid.

In some embodiments, the microbial oil has a similar ratio of saturated to unsaturated fatty acids as plant-derived palm oil. Some plant-derived palm oils have approximately 50% of each. In some embodiments, the microbial oil has a saturated fatty acid composition of about 50% and an unsaturated fatty acid composition of about 50%. In some embodiments, the microbial oil has a saturated fatty acid composition of about 40-60% and an unsaturated fatty acid composition of about 40-60%. In some embodiments, the microbial oil has a saturated fatty acid composition of about 30-70% and an unsaturated fatty acid composition of about 30-70%. In some embodiments, the microbial oil has about 30%, 35%, 40%, 45%, 50%, 55%, 60%, 65%, or 70% saturated fatty acids.

In some embodiments, the microbial oil has a similar level of mono-unsaturated fatty acids as plant-derived palm oil. Some plant-derived palm oils contain approximately 40% mono-unsaturated fatty acids. In some embodiments, the microbial oil contains about 40% mono-unsaturated fatty acids. In some embodiments, the microbial oil contains about 30-50% mono-unsaturated fatty acids. In some embodiments, the microbial oil contains about 5-60% mono-unsaturated fatty acids. In some embodiments, the microbial oil has about 5%, 10%, 15%, 20%, 25%, 30%, 35%, 40%, 45%, 50%, 55%, or 60% mono-unsaturated fatty acids.

In some embodiments, the microbial oil has a similar level of poly-unsaturated fatty acids as plant-derived palm oil. Some plant-derived palm oils contain approximately 10% poly-unsaturated fatty acids. In some embodiments, the microbial oil contains about 10% poly-unsaturated fatty acids. In some embodiments, the microbial oil contains about 5-25% poly-unsaturated fatty acids. In some embodiments, the microbial oil has about 5%, 6%, 7%, 8%, 9%, 10%, 11%, 12%, 13%, 14%, 15%, 16%, 17%, 18%, 19%, 20%, 21%, 22%, 23%, 24%, or 25% poly-unsaturated fatty acids.

In some embodiments, the microbial oil has a similar iodine value as plant-derived palm oil. Some plant-derived palm oils have an iodine value of about 50.4-53.7. In some embodiments, the microbial oil has an iodine value of about 49-65. In some embodiments, the microbial oil has an iodine value of about 49, 50, 51, 52, 53, 54, 55, 56, 57, 58, 59, 60, 61, 62, 63, 64, or 65.

Table 1 shows ranges for the fatty acid composition of an illustrative plant-derived palm oil and ranges of values for the fatty acid composition of illustrative microbial oil. In some embodiments, the microbial oil has one or more fatty acid composition parameters similar to those of Table 1. For example, in some embodiments, the microbial oil has a value within the plant-derived palm oil range for a given fatty acid composition parameter. In some embodiments, the microbial oil has a value within the microbial oil ranges provided in Table 1 for one or more parameters.

TABLE 1
Illustrative fatty acid compositions of microbial oil

| | Illustrative | Illustrative |
|---|---|---|
| | plant-derived | microbial |
| Component | palm oil range | oil range |
| C8:0 | 0.0-0.1% | 0.0% |
| C10:0 | 0.0-0.1% | 0.0-0.1% |
| C12:0 | 0.0-0.5% | 0.0-0.5% |
| C14:0 | 0.5-2.0% | 0.0-5.0% |
| C14:1c | 0.0-0.1% | 0.0-0.2% |
| C15:1 | 0.0-0.1% | 0.0-1.0% |
| C16:0 | 39.3-47.5% | 10.0-50.0% |
| C16:1 | 0.0-0.6% | 0.0-1.0% |
| C17:0 | 0.0-0.2% | 0.0-15.0% |
| C17:1 | 0.0-0.1% | 0.0-0.1% |
| C18:0 | 3.5-6.0% | 7.0-35.0% |
| C18:1 | 36.0-44.0% | 10.0-50.0% |
| C18:2 | 9.0-12.0% | 8.0-20.0% |
| C18:3 | 0.0-0.5% | 0.0-0.5% |
| C20:0 | 0.0% | 0.0-10.0% |
| C20:1 | 0.0-0.4% | 0.0-5.0% |
| C22:0 | 0.0-0.2% | 0.0-5.0% |
| C22:1 | 0.0% | 0.0-1.0% |
| C22:2 | 0.0% | 0.0-5.0% |
| C24:0 | 0.0% | 0.0-10.0% |

Tables 2A and 2B show ranges for the triglyceride composition of an illustrative plant-derived palm oil and ranges of values for the triglyceride composition of illustrative microbial oil. The abbreviations used are as follows: S: Stearic fatty acid; P: Palmitic fatty acid; O: Oleic fatty acid. For each component shown below in Table 2A, for example P-O-P, the corresponding measurements for that molecule may also include other isomers, for example P-P-O and O-P-P. In some embodiments, the microbial oil has one or more triglyceride composition parameters similar to those of Table 2A and Table 2B. For example, in some embodiments, the microbial oil has a value similar to or within the plant-derived palm oil range for a given triglyceride composition parameter. For example, plant-derived palm oil has an O-O-P of approximately 23.24% and microbial-derived oil has an O-O-P of approximately 20.78. In some embodiments, the microbial oil has a similar triglyceride content to that of plant-derived palm oil. For example, the total triglyceride content of sat-unsat-sat in plant-derived palm oil is approximately 49.53 and microbial-derived oil has approximately 49.42. In some embodiments, the microbial oil has a value different than plant-derived palm oil. For example, plant-derived palm oil has approximately 9.04% sat-sat-sat chains, whereas microbial-derived oil has approximately 3.36%. Some plant-derived palm oils have a triglyceride content of over 95%. In some embodiments, the microbial oil has a triglyceride content of 90-98%. In some embodiments, the microbial oil has a triglyceride content of about 90, 91, 92, 93, 94, 95, 96, 97, or 98%.

TABLE 2A
Illustrative triglyceride compositions of microbial oil

| | Crude | Crude |
|---|---|---|
| | plant-derived | microbial |
| Component | palm oil range | oil range |
| P-P-P | 6.48 +/− 1.62 | 1.02 +/− 0.25 |
| P-P-O | 31.62 +/− 7.9 | 22.53 +/− 5.63 |
| O-O-P | 23.24 +/− 5.81 | 20.78 +/− 5.12 |
| S-O-S | 0.6 +/− 0.15 | 1.53 +/− 0.38 |
| S-O-O | 2.46 +/− 0.62 | 4.29 +/− 1.07 |
| P-O-S | 6.11 +/− 1.53 | 10.25 +/− 2.56 |
| M-O-P | 1.58 +/− 0.40 | 4.73 +/− 1.18 |
| Sat-Sat-Sat | 9.04 +/− 1.36 | 3.36 +/− 0.50 |
| Sat-Unsat-Sat | 49.53 +/− 7.43 | 49.42 +/− 7.41 |
| Sat-Unsat-Unsat | 36.66 +/− 5.50 | 39.42 +/− 5.91 |
| Unsat-Unsat-Unsat | 4.77 +/− 0.72 | 6.86 +/− 1.03 |

TABLE 2B
Summary total triglyceride compositions

| | Number of unsaturated | | | | |
|---|---|---|---|---|---|
| | side chains | | | | |
| | 0 | 1 | 2 | 3 | total |
| Crude Plant-derived | 9.04% | 49.53% | 36.66% | 4.76887% | 100.00 |
| palm oil | | | | | |
| Crude Microbial- | 3.36% | 49.42% | 39.42% | 6.86% | 99.06 |
| derived oil | | | | | |

In some embodiments, the microbial oil has a similar diacylglycerol content as a plant-derived palm oil. Percentage of diacylglycerol varies between about 4-11% for some plant-derived palm oils. In some embodiments, the microbial oil comprises 0-15% diacylglycerol content.

In some embodiments, the microbial oil has a similar triacylglycerol profile to plant-derived palm oil. Some plant-derived palm oils have over 80% C50 and C52 triacylgylcerols. In some embodiments, the microbial oil has a triacylglycerol profile comprising at least 40% C50 and C52 triacylglycerols.

In some embodiments, the microbial oil has a similar slip melting point to plant-derived palm oil. Some plant-derived palm oils have a slip melting point of about 33.8-39.2° C. In some embodiments, the microbial oil has a slip melting point of about 30-40° C. In some embodiments, the microbial oil has a slip melting point of about 30, 31, 32, 33, 34, 35, 36, 37, 38, 39, or 40° C.

In some embodiments, the microbial oil has a saponification value similar to that of plant-derived palm oil. Some plant-derived palm oils have a saponification value of about 190-209. In some embodiments, the microbial oil has a saponification value of about 150-210. In some embodiments, the microbial oil has a saponification value of about 150, 155, 160, 165, 170, 175, 180, 185, 190, 195, 200, 205, or 210.

In some embodiments, the microbial oil has a similar unsaponifiable matter content to that of plant-derived palm oil. Some plant-derived palm oils have an unsaponifiable matter content of about 0.19-0.44% by weight. In some embodiments, the microbial oil has an unsaponifiable matter content of less than 5% by weight.

In some embodiments, the microbial oil has a similar refractive index to that of plant-derived palm oil. Some plant-derived palm oils have a refractive index of about 1.4521-1.4541. In some embodiments, the microbial oil has a refractive index of about 1.3-1.6.

In some embodiments, the microbial oil has a similar apparent density to that of plant-derived palm oil. Some plant-derived palm oils have an apparent density of about 0.8889-0.8896. In some embodiments, the microbial oil has an apparent density of about 0.88-0.9.

In some embodiments, the microbial oil has one or more parameters similar to those of hybrid palm oil.

In some embodiments, the microbial oil may be used as a palm oil substitute or alternative. In some embodiments, the microbial oil may be used in the manufacture of any product for which palm oil can be employed. For example, in some embodiments, the microbial oil may be used in the production of soap bases, detergents, and oleochemicals. In some embodiments, the microbial oil may be used in the production of food products.

### Processing of Microbial Oil

In some embodiments, once the microbial oil is obtained from the oleaginous microorganism, it is subjected to some form of processing. In some embodiments, the microbial oil is refined, bleached, deodorized, fractionated, treated, and/or derivatized.

In some embodiments, the microbial oil is refined. In some embodiments, prior to refinement, the microbial oil is referred to as crude microbial oil. In some embodiments, the refinement process comprises the removal of one or more non-triacylglycerol components. Typical non-triacylglycerol components removed or reduced via oil refinement include free fatty acids, partial acylglycerols, phosphatides, metallic compounds, pigments, oxidation products, glycolipids, hydrocarbons, sterols, tocopherols, waxes, and phosphorous. In some embodiments, refinement removes certain minor components of the crude microbial oil with the least possible damage to the oil fraction (e.g., trans fatty acids, polymeric and oxidized triacylglycerols, etc.) and minimal losses of desirable constituents (e.g., tocopherols, tocotrienols, sterols, etc.). In some embodiments, processing parameters are adapted for retention of desirable minor components like tocopherols and tocotrienols and minimal production of unwanted trans fatty acids. See Gibon (2012) “Palm Oil and Palm Kernel Oil Refining and Fractionation Technology,” incorporated by reference herein in its entirety, for additional details of oil processing that are useful for the present microbial oils.

Common processing methods include physical refining, chemical refining, or a combination. In some embodiments, chemical refining comprises one or more of the following steps: degumming, neutralization, bleaching and deodorization. In some embodiments, physical refining comprises one or more of the following steps: degumming, bleaching, and steam-refining deodorization. While “physical refining” and “chemical refining,” as used herein and in the art, may refer to a general process of oil purification comprising multiple steps, possibly including bleaching and/or deodorizing, in the context of the present disclosure, the term “refined” as it relates to a microbial oil, e.g., a refined microbial oil, refers to a microbial oil from which one or more impurities or constituents have been removed other than odor and pigment. As such, stating that a microbial oil is refined does not indicate that the microbial oil has been deodorized and/or bleached. The term “RBD,” as used herein and as applied to a microbial oil, indicates that the microbial oil has been each of refined, bleached, and/or deodorized.

In some embodiments, in chemical refining, the free fatty acids and most of the phosphatides are removed during alkali neutralization. In some embodiments, the non-hydratable phosphatides are first activated with acid and further washed out together with the free fatty acids during alkali neutralization with caustic soda. In some embodiments, chemical refining comprises one or more steps of acid treatment, centrifugation, bleaching, deodorizing, and the like.

In some embodiments, during physical refining, phosphatides are removed by a specific degumming process and the free fatty acids are distilled during the steam refining/deodorization process. In some embodiments, the degumming process is dry degumming or wet acid degumming. In some embodiments, physical refining is employed when the acidity of the crude microbial oil is sufficiently high. In some embodiments, physical refining is employed for crude microbial oil with high initial free fatty acid (FFA) content and relatively low phosphatides.

In some embodiments, the microbial oil is deodorized. In some embodiments, the deodorization process comprises steam refining. In some embodiments, deodorization comprises vacuum steam stripping at elevated temperature during which free fatty acids and volatile odoriferous components are removed to obtain bland and odorless oil. Optimal deodorization parameters (temperature, vacuum, and amount of stripping gas) are determined by the type of oil and the selected refining process (chemical or physical refining) but also by the deodorizer design.

In some embodiments, the microbial oil is bleached. In some embodiments, the bleaching is performed through the use of bleaching earth, e.g., bleaching clays. In some embodiments, the bleaching method employed is the two stage co-current process, the counter-current process, or the Oehmi process. In some embodiments, the bleaching method is dry bleaching or wet bleaching. In some embodiments, bleaching is accomplished through heat bleaching. In some embodiments, bleaching and deodorizing occur concurrently.

In some embodiments, the microbial oil is refined, bleached, and/or deodorized.

In some embodiments, the microbial oil is not bleached or is only partially bleached. For example, in some embodiments, the microbial oil still retains pigments after processing. In some embodiments, the microbial oil comprises any one or more of the pigments referenced herein. Therefore, in some embodiments, the microbial oil is refined and deodorized, but not bleached or not fully bleached.

In some embodiments, the microbial oil is processed and/or modified via one or more of fractionation, interesterification, trans-esterification, hydrogenation, steam hydrolysis, distillation, and saponification.

In some embodiments, the microbial oil is fractionated. In some embodiments, fractionation is carried out in multiple stages, resulting in fractions appropriate for different downstream indications. In some embodiments, the microbial oil is fractionated via dry fractionation. In some embodiments, the microbial oil is fractionated via wet fractionation. In some embodiments, the microbial oil is fractionated via solvent/detergent fractionation.

In some embodiments, the microbial oil is modified via interesterification. In some embodiments, the interesterification is enzymatic. In some embodiments, the interesterification is chemical.

In some embodiments, the microbial oil is derivatized. In some embodiments, the oil is derivatized to free fatty acids and glycerol. In some embodiments, the oil is derivatized to fatty alcohols. In some embodiments, the oil is derivatized to esters. In some embodiments, the oil is derivatized to fatty acid methyl esters (FAMEs).

The present description is made with reference to the accompanying drawings and Examples, in which various example embodiments are shown. However, many different example embodiments may be used, and thus the description should not be construed as limited to the example embodiments set forth herein. Rather, these example embodiments are provided so that this disclosure will be thorough and complete. Various modifications to the exemplary embodiments will be readily apparent to those skilled in the art, and the generic principles defined herein may be applied to other embodiments and applications without departing from the spirit and scope of the disclosure. Thus, this disclosure is not intended to be limited to the embodiments shown, but is to be accorded the widest scope consistent with the principles and features disclosed herein.

Although the disclosure may not expressly disclose that some embodiments or features described herein may be combined with other embodiments or features described herein, this disclosure should be read to describe any such combinations that would be practicable by one of ordinary skill in the art. Unless otherwise indicated herein, the term “include” shall mean “include, without limitation,” and the term “or” shall mean non-exclusive “or” in the manner of “and/or.”

Those skilled in the art will recognize that, in some embodiments, some of the operations described herein may be performed by human implementation, or through a combination of automated and manual means. When an operation is not fully automated, appropriate components of embodiments of the disclosure may, for example, receive the results of human performance of the operations rather than generate results through its own operational capabilities.

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world, or that they disclose essential matter.

### EXAMPLES

### Example 1

### Fatty Acid Composition of Exemplary Microbial Oil

To compare the fatty acid composition of an exemplary microbial oil to that of a plant-derived palm oil, the oil samples were converted into fatty acid methyl esters and then analyzed using gas chromatography-mass spectrometry (GC-MS).

### FAME Preparation

A method of using commercial aqueous concentrated HCl (conc. HCl; 35%, w/w) as an acid catalyst was employed for preparation of fatty acid methyl esters (FAMEs) from microbial oil and palm oil for GC-MS. FAME preparation was conducted according to the following exemplary protocol.

Commercial concentrated HCl (35%, w/w; 9.7 ml) was diluted with 41.5 ml of methanol to make 50 ml of 8.0% (w/v) HCl. This HCl reagent contained 85% (v/v) methanol and 15% (v/v) water that was derived from conc. HCl and was stored in a refrigerator.

A lipid sample was placed in a screw-capped glass test tube (16.5×105 mm) and dissolved in 0.20 ml of toluene. To the lipid solution, 1.50 ml of methanol and 0.30 ml of the 8.0% HCl solution were added in this order. The final HCl concentration was 1.2% (w/v) or 0.39 M, which corresponded to 0.06 ml of concentrated HCl in a total volume of 2 ml. The tube was vortexed and then incubated at 45° C. overnight (14 h or longer) for mild methanolysis/methylation or heated at 100° C. for 1 h for rapid reaction. After cooling to room temperature, 1 ml of hexane and 1 ml of water were added for extraction of FAMEs. The tube was vortexed, and then the hexane layer was analyzed by GC-MS directly or after purification through a silica gel column.

### GC-MS

For the analysis of fatty acid composition, a Shimadzu GCMS-TQ8040/GC-2010 Plus instrument was employed. The FAME samples were concentrated at 5 g/L in hexane/chloroform/heptane prior to analysis.

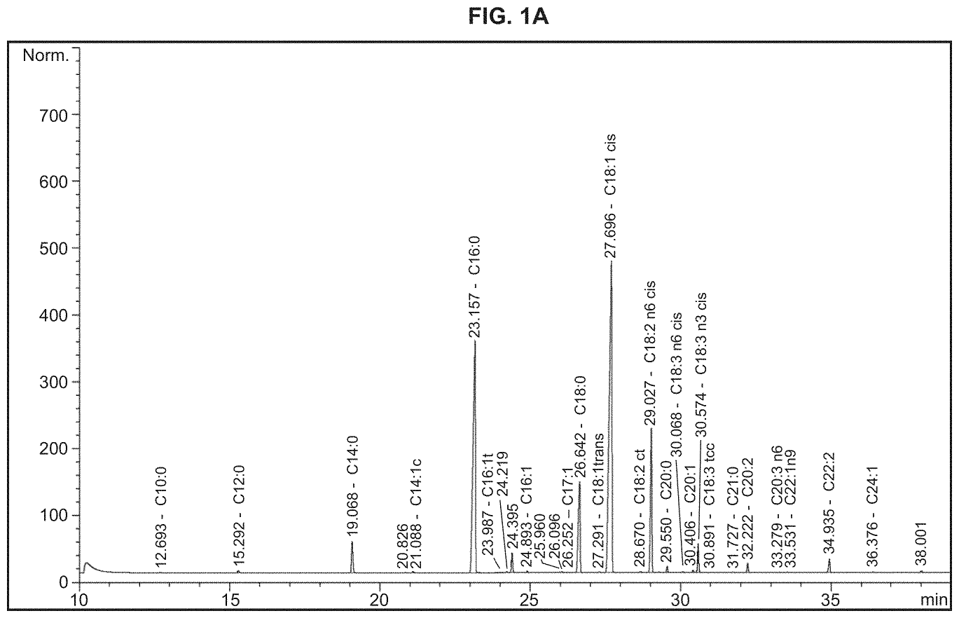

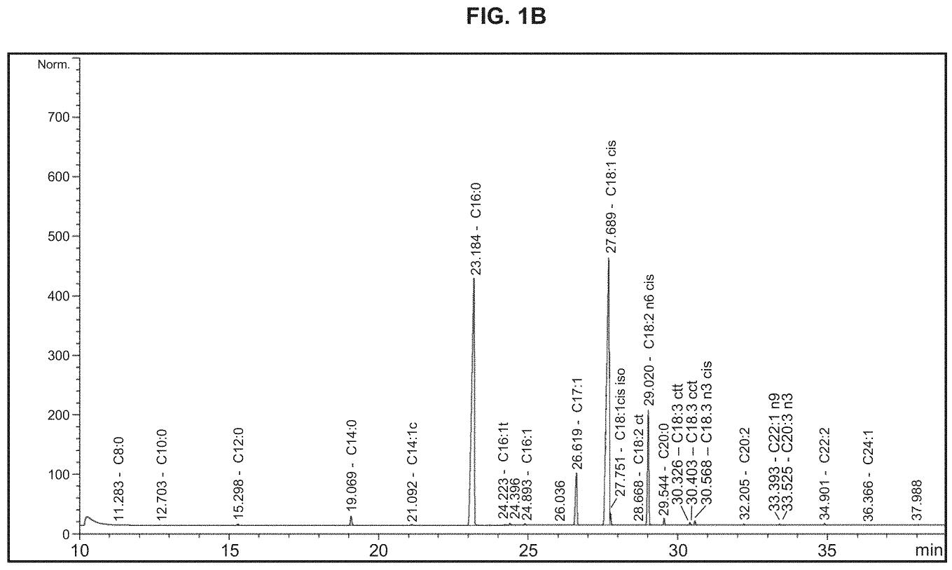

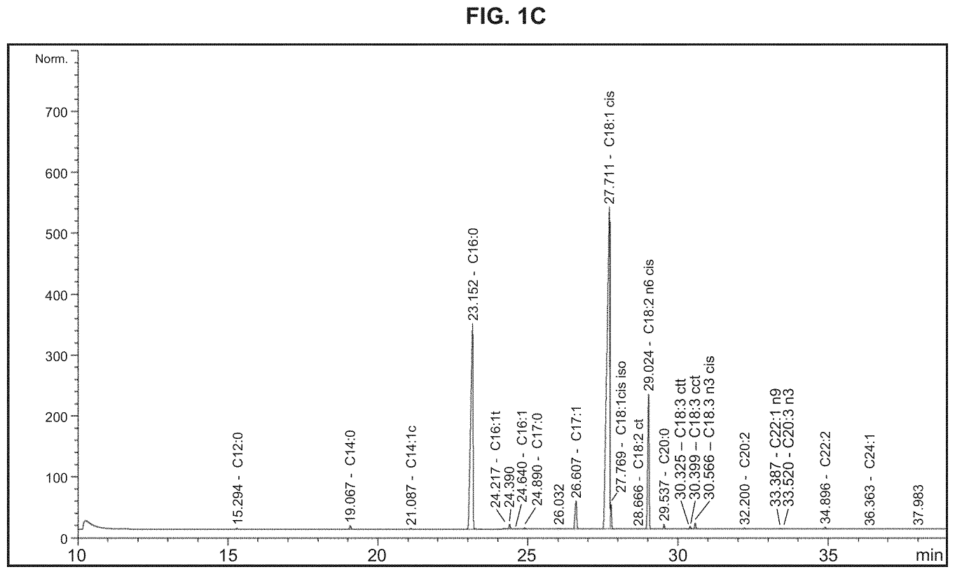

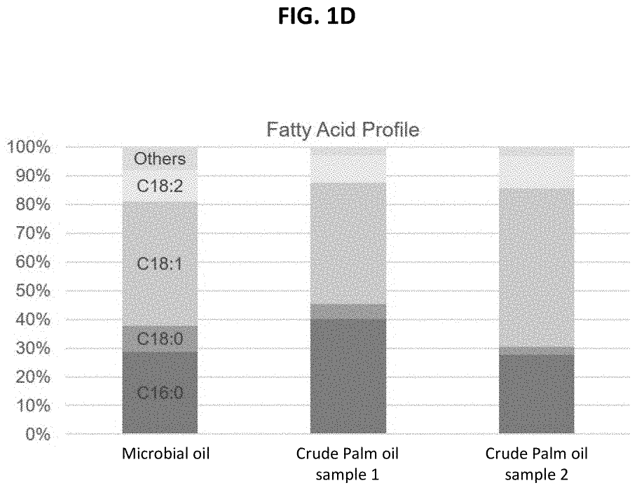

The results of the analysis are shown in Table 3 comparing the fatty acid composition of three exemplary microbial oil samples produced by Rhodosporidium toruloides to the measurements expected for crude palm oil, as set forth by guidelines from the Malaysian government. For Microbial oil sample 3, the fatty acid compositions were determined via fatty acid methyl ester analysis with a GC-SSL/FID (7890A, Agilent) instrument. The methods employed were using AOCS Ce 1a-13 and AOCS C2 2-66. (see also FIG. 1A-1D). Table 3 shows the breakdown of the individual fatty acid constituents by w/w percent, with the percentages for each sample adding up to 100%. Fatty acids that were assayed but not detected in any sample include C4, C6, C13, C15, C15:1, C18:2 tt, C18:2 5,9, C18:2 tc, C18:3, C18:3 ctc, C18:3 ttt, C18:3 ttc+tct, C20:4 n6ARA, C22, and C24.

TABLE 3
Fatty acid composition of microbial oil samples

| | Microbial | Microbial | Microbial | Palm | Palm |
|---|---|---|---|---|---|
| Fatty | oil | oil | oil | oil | oil |
| Acid | Sample 1 | Sample 2 | sample 3 | MIN | MAX |
| C8:0 | 0.0% | 0.0% | 0.0% | 0.0% | 0.1% |
| C10:0 | 0.0% | 0.0% | 0.04% | 0.0% | 0.1% |
| C12:0 | 0.2% | 0.0% | 0.17% | 0.0% | 0.5% |
| C14:0 | 1.8% | 1.7% | 2.24% | 0.5% | 2.0% |
| C15:1 | 0.5% | 0.5% | 0.0% | 0.0% | 0.1% |
| C16:0 | 14.5% | 13.8% | 28.7% | 39.3% | 47.5% |
| C16:1 | 0.6% | 0.7% | 0.10% | 0.0% | 0.6% |
| C17:0 | 10.2% | 9.5% | 0.0% | 0.0% | 0.2% |
| C17:1 | 0.8% | 0.6% | 0.03% | 0.0% | 0.1% |
| C18:0 | 26.9% | 28.8% | 8.98% | 3.5% | 6.0% |
| C18:1 | 10.0% | 16.3% | 43.39% | 36.0% | 44.0% |
| C18:2 | 15.2% | 16.1% | 10.77% | 9.0% | 12.0% |
| C20:0 | 8.3% | 3.6% | 0.0% | 0.0% | 0.0% |
| C18:3 | 0.2% | 0.0% | 1.75% | 0.0% | 0.5% |
| C20:1 | 2.5% | 0.4% | 0.13% | 0.0% | 0.4% |
| C22:0 | 2.6% | 0.7% | 0.0% | 0.0% | 0.2% |
| C22:1 | 0.3% | 0.3% | 0.02% | 0.0% | 0.0% |
| C22:2 | 0.3% | 0.0% | 0.94% | 0.0% | 0.0% |
| C24:0 | 5.0% | 7.1% | 0.0% | 0.0% | 0.0% |
| Other | | | 2.74% | | |

These results show that exemplary microbial oil samples of the present disclosure have a similar breakdown of saturated vs. unsaturated fatty acids compared to plant-derived palm oil, though the specific identities of the predominant fatty acids differs between the microbial samples and typical palm oil. Similar to palm oil, though, C16:0 was a significant source of saturated fatty acid in the microbial samples and C18 unsaturated fatty acids made up the majority of the unsaturated fatty acids in the sample.

The fatty acid composition breakdown of the samples were determined via fatty acid methyl ester analysis with a GC-SSL/FID (7890A, Agilent) instrument. The methods employed were using AOCS Ce 1a-13 and AOCS C2 2-66. The results these analyses are shown in Table 4 and FIG. 1A-1C. Table 4 below shows the breakdown of the individual fatty acid constituents by w/w percent, with the percentages for each sample adding up to 100%. Fatty acids that were assayed but not detected in any sample include C4, C6, C13, C15, C15:1, C18:2 tt, C18:2 5,9, C18:2 tc, C18:3, C18:3 ctc, C18:3 ttt, C18:3 ttc+tct, C20:4 n6ARA, C22, and C24.

TABLE 4
Fatty acid composition breakdown

| | | Crude | Crude | Crude |
|---|---|---|---|---|
| Condensed | Common | microbial | palm | hybrid |
| formula | name | oil | oil | palm oil |
| C8:0 | Caprylic | | 0.01% | |
| C10:0 | Capric | 0.04% | 0.01% | |
| C11:0 | Undecylic | 0.00% | | |
| C12:0 | Lauric | 0.17% | 0.11% | 0.08% |
| C14:0 | Myristic | 2.24% | 0.75% | 0.27% |
| C14:1c | Myristoleic | 0.08% | 0.05% | 0.06% |
| C16:0 | Palmitic | 28.70% | 40.20% | 27.79% |
| C16:1t | | 0.01% | 0.04% | 0.05% |
| C16:1 | Palmitoleic | 0.10% | 0.10% | 0.01% |
| C17:0 | Margaric | | | 0.08% |
| C17:1 | | 0.03% | | |
| C18:0 | Stearic | 8.98% | 5.15% | 2.65% |
| C18:1trans | | 0.08% | | |
| C18:1 cis | Oleic | 43.39% | 42.09% | 55.21% |
| C18:1cis iso | | | 0.59% | 1.11% |
| C18:2 ct | | 0.07% | 0.01% | 0.01% |
| C18:2 n6 cis | Linoleic | 10.77% | 9.61% | 11.23% |
| C18:3 ctt | | | 0.01% | 0.01% |
| C20:0 | Arachidic | 0.35% | 0.41% | 0.28% |
| C18:3 cct | | | 0.15% | 0.15% |
| C18:3 n6 cis | γ-Linolenic | 0.05% | | |
| | (GLA) | | | |
| C18:3 tcc | | 0.01% | | |
| C20:1 | | 0.13% | | |
| C18:3 n3 cis | α-Linolenic | 1.69% | 0.30% | 0.39% |
| | (ALA) | | | |
| C21:0 | heneicosylic | 0.03% | | |
| C20:2 | cis-11,14- | 0.54% | 0.07% | 0.06% |
| | eicosadienoic | | | |
| C20:3 n6 | | 0.02% | | |
| C22:1n9 | Erucic | 0.02% | 0.05% | 0.04% |
| C20:3 n3 | | | 0.02% | 0.02% |
| C22:2 | | 0.94% | 0.09% | 0.10% |
| C24:1 | | 0.04% | 0.01% | 0.01% |
| Unknown | | 1.20% | | |

Table 5 shows the w/w percentage of saturate, trans, mono-unsaturated, poly-unsaturated, and unknown fatty acids in each sample. The fatty acid compositions were determined via fatty acid methyl ester analysis with a GC-SSL/FID (7890A, Agilent) instrument. The methods employed were using AOCS Ce 1a-13 and AOCS C2 2-66. FIG. 1A-1C show the chromatograms for the crude microbial oil (FIG. 1A), palm oil (FIG. 1B), and hybrid palm oil (FIG. 1C), respectively. FIG. 1D shows a bar graph of representative compositions of microbial oil and palm oil.

TABLE 5
Overall fatty acid composition

| | Crude | Crude | Crude |
|---|---|---|---|
| | microbial | palm | hybrid |
| | oil | oil | palm oil |
| Saturated fatty acid | 40.5% | 41.5% | 28.5% |
| Trans fatty acid | 0.17% | 0.21% | 0.22% |
| Mono-unsaturated fatty | 43.8% | 48% | 59.1% |
| acid | | | |
| Poly-unsaturated fatty | 14% | 10.1% | 11.8% |
| acid | | | |
| Unknown | 1.5% | 0% | 0% |

### Example 2

### Fractionation and Saturation Analysis of Exemplary Microbial Oil Composition

Fats and oils are mixtures of hydrocarbons of various chain lengths and saturation levels. Fractionation may be used to physically separate room temperature oil into saturated and unsaturated components. The melting points of full oil mixtures and their saturated/unsaturated components differ. Hydrophilization makes use of surface active agents (surfactants) that dissolve solidified fatty crystals and emulsify liquid oils. By centrifuging this hydrophilized suspension, fats can be separated into different fractions based on saturation. A palm oil and a microbial oil were fractionated and the saturation levels of their fractions were compared.

### Fractionation

Crude palm oil and an R. toruloides microbial oil were fractionated using a method as set out in, e.g., Stein, W., “The Hydrophilization Process for the Separation of Fatty Materials,” Henkel and Cie, GmbH, Presented at AOCS Meeting, New Orleans, May 1967.

The oil sample was weighed and then incompletely melted to 50° C. The temperature was then brought down to 32° C. over the course of 10 min. The temperature was then slowly lowered to 20° C. with periods of time held at select temperatures between 32° C.-20° C. as follows: 32° C.—30 min; 26° C.—15 min; 24° C.—15 min; 22° C.—15 min; 21° C.—15 min; 20° C.—15 min. The oil sample was then maintained at 20° C. for an additional 1 hr.

After this temperature manipulation, the oil sample was emulsified in a wetting agent solution at a ratio of 1:1.5 w/w fat to wetting agent. The wetting agent was comprised of a salt and a detergent in DI water: 0.3% (w/w) sodium lauryl sulfate; 4% (w/w) magnesium sulfate. The oil/wetting agent mixtures were vortexed until thoroughly mixed. The samples were centrifuged at 4700 rpm for 5 min in a benchtop centrifuge. The lighter oil phase migrated to the top, while the heavier aqueous phase (containing solid, saturated fatty particles) migrated to the bottom. The aqueous phase was separated by aspirating the upper olein phase into a pre-weighed scintillation vial. The aqueous phase was heated—with its solidified stearin layer interspersed atop—until all fatty materials melted. This heated aqueous phase was centrifuged (4700 rpm, 1 min, 40° C.) and the stearin fraction was also aspirated into a pre-weighed scintillation vial.

The separated olein and stearin fractions were weighed and their masses compared to the original mass of oil pre-fractionation. By mass, an exemplary microbial oil produced by R. toruloides was 68.4% w/w olein and 31.6% w/w stearin. By comparison, a crude plant-derived palm oil sample was analyzed as comprising 72% w/w olein and 28% w/w stearin using this fractionation method.

### Saturation Level Measurement

Next, the iodine value (IV) for each fraction was calculated, which is expressed as the number of grams of iodine absorbed by 100 g of the oil sample. The microbial olein fraction had an iodine value of 81 and the microbial stearin fraction had an iodine value of 22. The crude palm oil olein fraction had an IV of 53 and the stearin fraction had an IV of 40. These results indicate an even more distinct fractionation of saturated and unsaturated fatty acids between the microbial fractions, a distinction that could be useful for the manufacture of downstream products, as plant-derived palm oil may require multiple fractionation steps to achieve this level of differentiation between fractions.

### Example 3

### Comprehensive Analysis of an Illustrative Crude Microbial Oil Sample

A 100 g sample of crude microbial oil produced by the oleaginous microorganism R. toruloides was analyzed for general physical chemical characterization; fatty acid content; triglyceride composition; unsaponifiable lipid content; oxidative stability; FAs at Sn-2 position; and contaminant (3-MCPD, GEs) levels. These analyses were carried out in comparison to standard Colombian palm oil and hybrid palm oil samples over the course of 70 days. Samples were stored in the dark, at cold temperatures, and at atmospheric nitrogen conditions.

### General Physical Chemical Characterization

The three oil samples were analyzed along different physical and chemical parameters, the results of which analyses are shown in Table 6. The methods employed were those of the American Oil Chemists' Society (AOCS) and are referenced within the Table by their AOCS identifier.

TABLE 6
General physical chemical characterization

| | | | | Crude | Crude | Crude |
|---|---|---|---|---|---|---|
| | | | | microbial | palm | hybrid |
| Parameter | Unit | Method | Equipment | oil | oil | palm oil |
| Free fatty acid | % | AOCS Ca | 865Dosimat plus | 2.58 | 2.81 | 2.02 |
| content | | 5a-40 | (Metrohm) | | | |
| Triglyceride | % | Arithmetical | — | 96.5 | 96.3 | 93.6 |
| content | | calculation | | | | |
| Diglyceride | % | AOCS Cd | GC-COC/FID | 0.94 | 5.49 | 4.04 |
| content | | 11b-91 | (7890A, Agilent) | | | |
| Monoglyceride | % | AOCS Cd | GC-COC/FID | <0.1 | <0.1 | <0.1 |
| content | | 11b-91 | (7890A, Agilent) | | | |
| Slip melting | ° C. | AOCS Cc | Magnetic Stirrer | <15 | 36.2 | <15 |
| point | | 3-25 | (MR-Hei-Std, | | | |
| | | | Heidolph) | | | |
| Color | red | AOCS Cc | Spectrocolorimeter | 46 | 28.4 | 39 |
| (Lovibond). | yellow | 13e−92 | PFXi Series | 70 | 47 | 70 |
| Day 0. | | (cuvette 1″) | 995 (Lovibond) | | | |

As shown in Table 6 above, crude microbial oil has similar amounts of free fatty acids, triglycerides, and monoglyceride as those found in crude palm oil and crude hybrid oil. Specific triglycerides were also measured and shown below.

### Triglyceride Composition

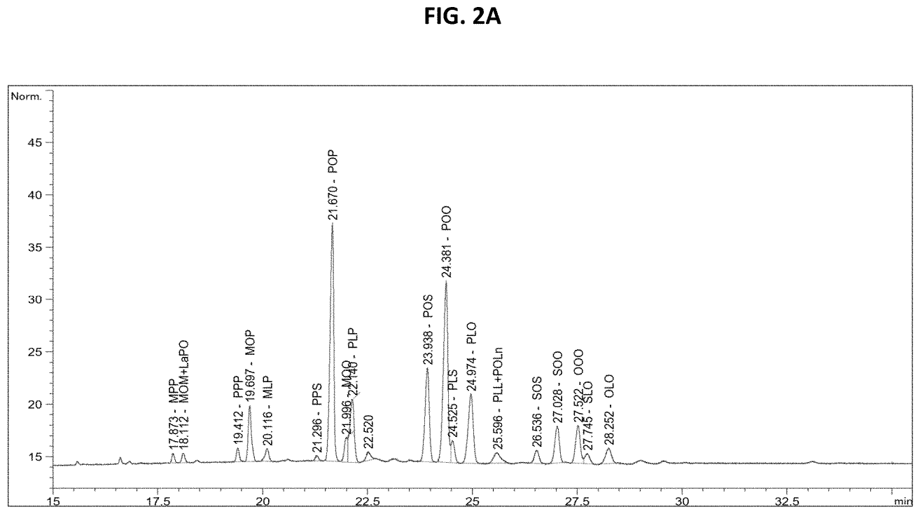

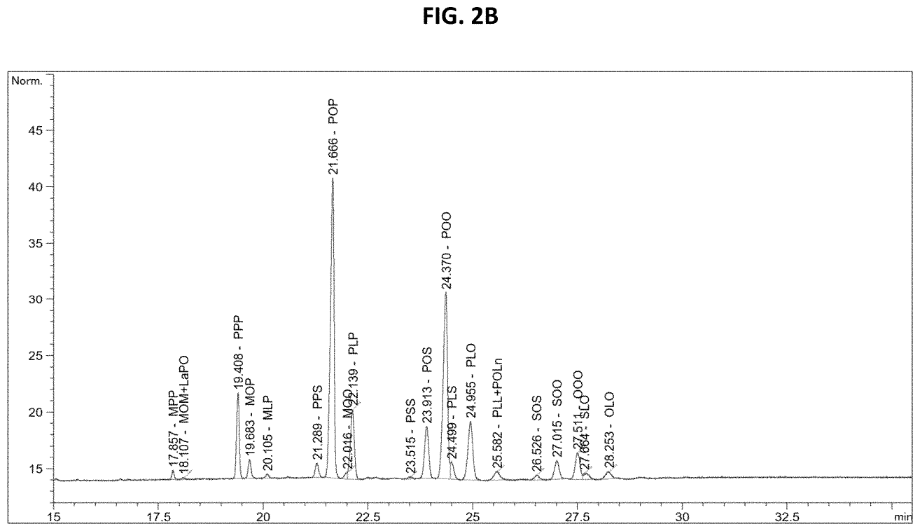

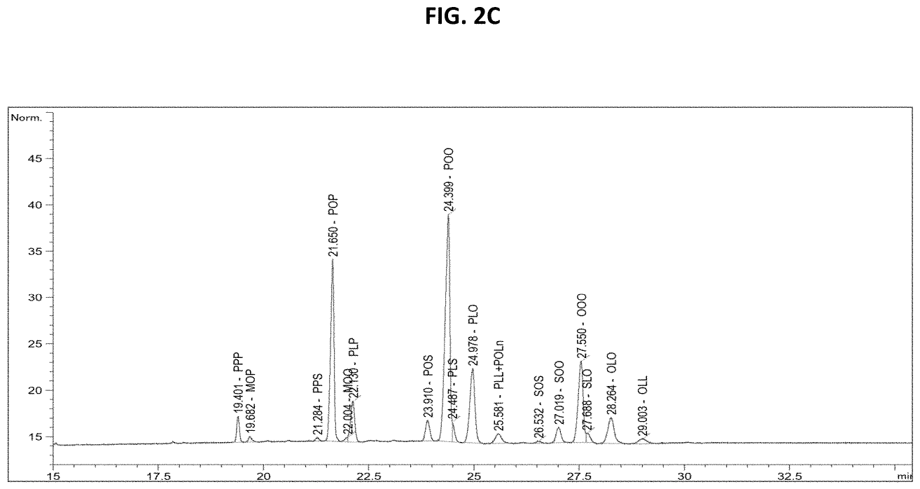

The triglyceride compositions of the three samples were analyzed on a GC-COC/FID (7890A, Agilent) instrument according to the AOCS Ce 5-86 method. Table 7 shows the results of the triglyceride analysis, with values as w/w percentages. The abbreviations used are as follows. M: Myristic fatty acid; S: Stearic fatty acid; P: Palmitic fatty acid; O: Oleic fatty acid; L: Linoleic fatty acid; La: Lauric fatty acid; Ln: linoleic fatty acid. The chromatogram for crude microbial oil is shown in FIG. 2A, the chromatogram for crude palm oil is shown in FIG. 2B, and the chromatogram for crude hybrid palm oil is shown in FIG. 2C.

TABLE 7
Triglyceride composition

| | | Crude | Crude | Crude |
|---|---|---|---|---|
| | | microbial | palm | hybrid |
| Triglyceride | Unit | oil | oil | palm oil |
| MPP | % | 0.65 | 0.60 | 0.00 |
| MOM + LaPO | % | 0.75 | 0.12 | 0.00 |
| PPP | % | 1.02 | 6.48 | 2.11 |
| MOP | % | 4.73 | 1.58 | 0.55 |
| MLP | % | 1.27 | 0.35 | 0.00 |
| PPS | % | 0.43 | 1.38 | 0.35 |
| POP | % | 22.53 | 31.62 | 19.45 |
| MOO | % | 1.89 | 0.49 | 0.37 |
| PLP | % | 7.51 | 7.87 | 5.20 |
| PSS | % | 0.00 | 0.23 | 0.00 |
| POS | % | 10.25 | 6.11 | 2.68 |
| POO | % | 20.78 | 23.24 | 32.62 |
| PLS | % | 2.12 | 1.62 | 1.38 |
| PLO | % | 9.11 | 8.08 | 11.53 |
| PLL + POLn | % | 2.04 | 1.41 | 1.78 |
| SSS | % | 0.00 | 0.00 | 0.00 |
| SOS | % | 1.53 | 0.60 | 0.29 |
| SOO | % | 4.29 | 2.46 | 2.29 |
| OOO | % | 4.54 | 3.63 | 12.17 |
| SLO | % | 1.30 | 0.98 | 1.09 |
| OLO | % | 2.33 | 1.14 | 4.93 |
| OLL | % | 0.00 | 0.00 | 1.23 |
| LLL | % | 0.00 | 0.00 | 0.00 |
| LLnL | % | 0.00 | 0.00 | 0.00 |
| LnLLn | % | 0.00 | 0.00 | 0.00 |
| LnLnLn | % | 0.00 | 0.00 | 0.00 |
| OOA | % | 0.00 | 0.00 | 0.00 |
| LLnLn | % | 0.00 | 0.00 | 0.00 |
| SOA | % | 0.00 | 0.00 | 0.00 |
| Total | % | 99.06219 | 100 | 100 |

The microbial oil sample showed similarity to both palm oil and hybrid palm oil along different parameters of fatty acid and triglyceride content. For example, microbial oil comprised approximately 1.2% w/w palmitic-palmitic-palmitic triglycerides, approximately 22.53% w/w palmitic-palmitic-oleic triglycerides, approximately 20.78% w/w oleic-oleic-palmitic triglycerides, approximately 1.53% w/w stearic-stearic-oleic triglycerides, and approximately 4.29% w/w stearic-oleic-oleic triglycerides.

### Fatty Acids at Sn-2 Position

The three samples were analyzed for the amount of palmitic and stearic fatty acids located at the sn-2 position of triglyceride molecules, with results shown in Table 8. Methods used were adapted from Luddy et al., “Pancreatic lipase hydrolysis of triglycerides by a semimicro technique,” Journal of the American Oil Chemists' Society 1964; 41(10):693-6, and Pina-Rodriguez et al., “Enrichment of amaranth oil with ethyl palmitate at the sn-2 position by chemical and enzymatic synthesis,” Journal of Agricultural and Food Chemistry 2009; 57(11):4657-62, each incorporated herein by reference in its entirety.

TABLE 8
Fatty acids at sn-2 position of triglycerides

| | | Crude | Crude | Crude |
|---|---|---|---|---|
| | | microbial | palm | hybrid |
| Parameter | Equipment | oil | oil | palm oil |
| Palmitic acid (%) | TLC silica gel 60 F254 | 12 | 14.4 | NA |
| at sn-2 position | GC-SSL/FID | | | |
| | (7890A, Agilent) | | | |
| Stearic acid (%) | | 12 | 14.1 | NA |
| at sn-2 position | | | | |

The microbial oil sample contained an acceptable amount of palmitic and stearic fatty acids located at the sn-2 position of the triglyceride molecules, suggesting the oil has suitability for use in various food products.

### Unsaponifiable Lipid Content

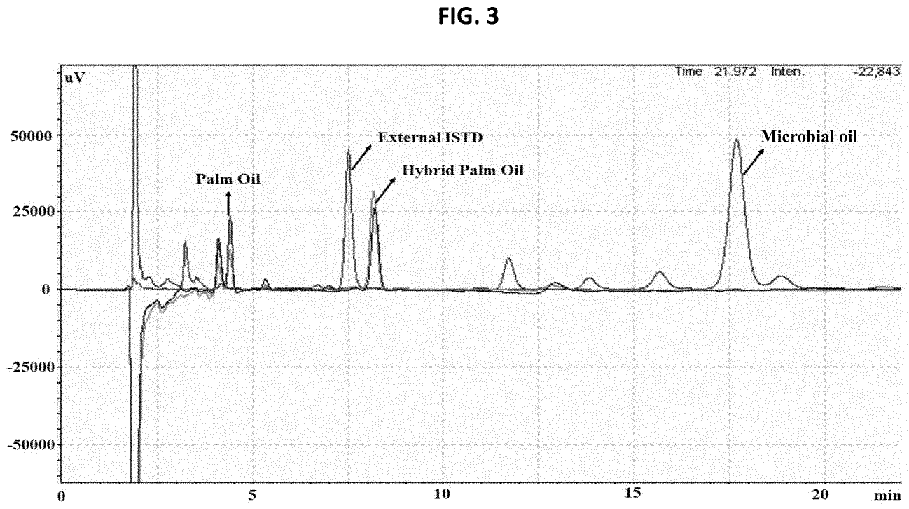

The unsaponifiable lipid content of the three samples was analyzed, specifically measuring the amount of β-carotene (data not shown), squalene, tocopherols, and sterols in each sample. Results are shown in Table 8. β-carotene was analyzed using the method of Luterotti et al., “New simple spectrophotometric assay of total carotenes in margarines,” Analytica Chimica Acta 2006; 573:466-473, incorporated by reference herein in its entirety. The sterol composition was analyzed using the method of Johnsson et al., “Side-chain autoxidation of stigmasterol and analysis of a mixture of phytosterol oxidation products by chromatographic and spectroscopic methods,” Journal of the American Oil Chemists' Society 2003; 80(8):777-83, incorporated by reference herein in its entirety, with the HPLC-DAD chromatogram results shown in FIG. 3. The other methods that were employed are indicated in Table 9. The sterol composition of the microbial oil sample showed an atypical sterols chromatographic profile differentiating it from the palm oil and hybrid palm oil samples and warranting further investigation. In this illustrative sample, the unexpected sterol composition acts as a unique fingerprint for the microbial oil sample.

TABLE 9
Unsaponifiable lipid content

| | | | Crude | Crude | Crude |
|---|---|---|---|---|---|
| | | | microbial | palm | hybrid |
| Parameter | Method | Equipment | oil | oil | palm oil |
| Squalene | AOCS | GC- | 122 | 389 | 260 |
| (ppm) | Ce | SSL/FID | | | |
| | 1a-13 | (7890A, | | | |
| | | Agilent) | | | |
| Tocopherols | AOCS | LC- | <10 | 869 | 761 |
| (ppm) | Ce | DAD/RID | | | |
| | 8-89 | (Prominence, | | | |
| | | Shimadzu) | | | |
| Sterols | Johnsson | GC- | Unexpected | 0.07 | 0.1 |
| (%) | et al. | COC/FID | profile | | |
| | | (7890A, | | | |
| | | Agilent) | | | |

As shown in Table 9, the microbial oil sample does not contain significant levels of unsaponifiable lipids, or tocopherols. Specifically, microbial oil has approximately 122 ppm of squalene, compared to 389 ppm and 260 ppm in palm oil and hybrid palm oil respectively. Microbial oil also contained less than 10 ppm of tocopherols, whereas palm oil and hybrid palm oil contained 869 ppm and 761 ppm respectively.

### Oxidative Stability

The oxidative stability of the samples was analyzed (data not shown) via The Ferric Reducing Ability of Plasma (FRAP) using the method of Szydłowska-Czerniak et al., “Effect of refining processes on antioxidant capacity, total contents of phenolics and carotenoids in palm oils,” Food Chemistry 2011; 129(3):1187-92, herein incorporated by reference in its entirety.

### Contaminant (3-MCPD, GEs, and Phosphorus) Levels

Levels of contaminants were assessed in each sample, with results shown in Table 10. The methods and equipment are shown in columns two and three, respectively.

TABLE 10
Contaminant levels

| | | | Crude | Crude | Crude |
|---|---|---|---|---|---|
| | | | microbial | palm | hybrid |
| Contaminant | Method | Equipment | oil | oil | palm oil |
| 3-MCPD | DGF | GC- | <LOQ | <LOQ | <LOQ |
| | C-VI | SSL/MSD | | | |
| | 18 (10) | (7890-5977A, | | | |
| | | Agilent) | | | |
| GEs | DGF | GC- | <LOQ | <LOQ | <LOQ |
| | C-VI | SSL/MSD | | | |
| | 18 (10) | (7890-5977A, | | | |
| | | Agilent) | | | |
| Phosphorus | AOCS | Spectro- | <1 ppm | 25 ppm | 20 ppm |
| content | Ca | photometer | | | |
| | 12-55 | UV-1280 | | | |
| | | (Shimadzu) | | | |

All three samples had contaminant levels below the limit of quantitation (LOQ). However, the samples differed greatly in the amount of phosphorous detected. Unlike crude palm oil and crude hybrid palm oil, which had 25 ppm and 20 ppm respectively, crude microbial oil had less than 1 ppm of phosphorous.

### Conclusion

Based on the above analyses, the crude microbial oil was a good match of palm oil/hybrid palm oil along a number of different parameters, demonstrating its suitability for use as an environmentally friendly alternative to plant-derived palm oil.

### Example 4

### Exemplary Microbial Oils from Three Different Strains of R. toruloides

### Fatty Acid Profile of Microbial Oil Produced by Three Exemplary Strains of Oleaginous Yeast

Using the FAME and GC-MS protocols of Example 1, exemplary microbial oils according to the present disclosure were analyzed from three illustrative strains of oleaginous yeast of the species Rhodosporidium toruloides: strain A, strain B, and strain C.

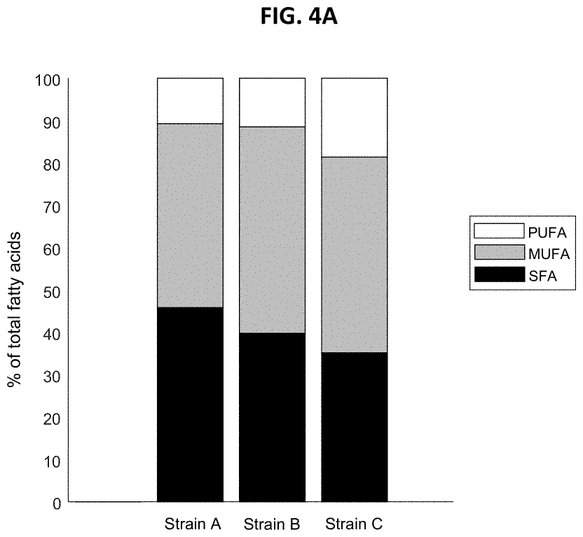

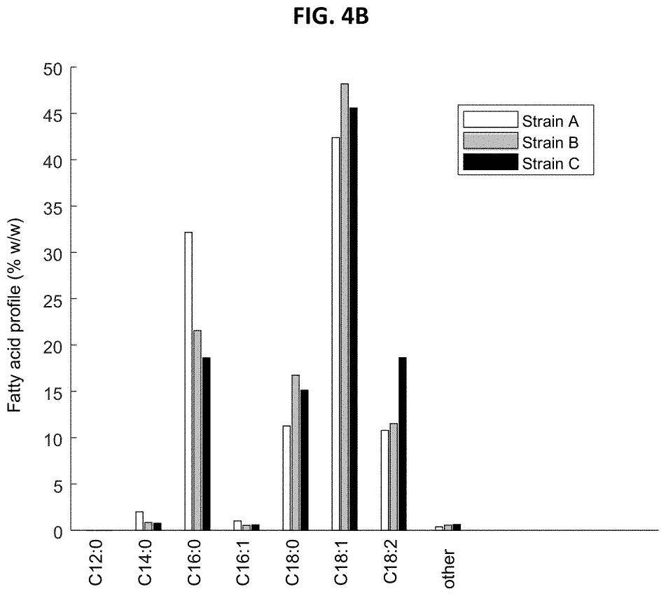

FIG. 4A shows the overall fatty acid composition broken down by percentage of poly-unsaturated fatty acid (PUFA), mono-unsaturated fatty acid (MUFA), and saturated fatty acid for exemplary microbial oils produced by these three strains. This breakdown shows a comparable ratio of saturated to unsaturated fatty acids within each sample, especially for strain A, which produced approximately equal amounts of saturated and unsaturated fatty acids. FIG. 4B shows the breakdown of the fatty acid composition for the microbial oils in terms of specific fatty acids. For all three microbial oils, C18:1 was most prevalent, comprising between 40-50% of each sample. The next most prevalent was C16:0, comprising 15-35% of each sample, followed by C18:0 and C18:2, which each made up about 10-20% of the samples. C14:0, C16:1, and C18:3 (not shown) each comprised less than 3% of the samples. The remaining less than 1% was made up of other fatty acids.

### Example 5

### Fractionation of Additional Exemplary Microbial Oils

### Fractionation Protocol

A 5 g sample of an exemplary R. toruloides microbial oil of the disclosure was melted to 50° C. over a hot plate. Temperature was brought down to 32° C. over 10 min and then slowly down to 20° C., allowing the sample to remain held at temperature every two degrees for 15 min. The sample was then held at 20° C. for 1 hr.

Wetting agent comprised of 0.3% (w/w) sodium lauryl sulfate and 4% (w/w) magnesium sulfate was added to the oil sample (1:1.5 w/w oil to wetting agent). The oil sample was vortexed thoroughly and then centrifuged at 4100 g for 5 min.

The liquid, upper lipid phase comprising a higher percentage of unsaturated fatty acids (olein) was transferred to a pre-weighed vial. The lower lipid phase (stearin), along with the remaining aqueous material, was heated until the stearin was fully melted. Then the sample was centrifuged for 1 min before the stearin layer was transferred to a separate pre-weighed vial. This process was repeated with a 10 g sample of crude palm oil.

### Effect of Fractionation on Fatty Acid Profile of Exemplary Microbial Oil

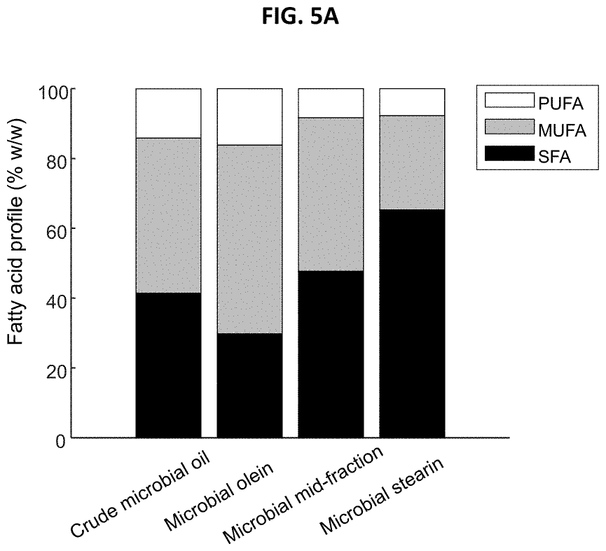

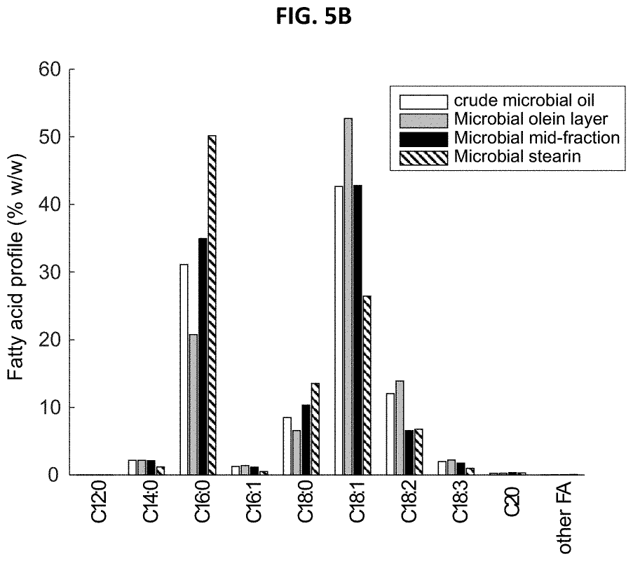

An exemplary R. toruloides microbial oil of the disclosure was fractionated. FIG. 5A shows the results of fractionation on overall fatty acid composition for a representative microbial oil. This figure demonstrates a higher percentage of unsaturated fatty acids in the olein fraction and a higher percentage of saturated fatty acids in the stearin fraction compared to the crude microbial oil. The microbial mid-fraction has a profile in between the olein and stearin profiles. FIG. 5B shows the breakdown in terms of specific fatty acids for the crude microbial oil and each of the fractions.

### Iodine Value Calculation

Iodine value was determined based on the Malaysian Palm Oil Board's test method. Briefly, approximately 0.5 g of oil was dissolved in 20 mL 1:1 cyclohexane/glacial acetic acid. 25 mL of Wijs reagent (iodine mono chloride dissolved in acetic acid) was added, and the solution was well stirred before being placed in the dark for 1 hr. A blank sample was prepared identically, without the addition of any oil sample.

At the end of the incubation time, 20 mL of 100 g/L potassium iodide and 150 mL of DI water were added. A standard volumetric solution of 0.1M sodium thiosulfate was added in a dropwise fashion until the solution's yellow color began to fade. 5 g/L starch solution was added until the solution turned a deep blue color. Additional thiosulfate titrant is added until the solution became clear upon mixing. The blank solution was titrated in parallel. For some samples, Metrohm's 892 professional rancimat was also used to confirm iodine values, in which case the starch solution was no longer needed as an indicator.

Iodine value was calculated as IV=12.69×C×(V1-V2)/M, where C is the concentration of sodium thiosulfate, V1 is the volume in mL of sodium thiosulfate used for the blank test, V2 is the volume in mL of sodium thiosulfate used for the determination, and M is the mass in g of the test oil sample.

### Effect of Fractionation on Iodine Value (IV) for an Exemplary Microbial Oil

The effect of fractionation on iodine value was evaluated using the protocol above for an illustrative crude R. toruloides microbial oil of the disclosure, along with its stearin and olein fractions. The results are summarized in Table 11 below.

TABLE 11
IVs for an exemplary fractionated microbial oil of the disclosure.

| | IV, replicate 1 | IV, replicate 2 |
|---|---|---|
| Sterol | (g/100 g fat) | (g/100 g fat) |
| Crude microbial oil | 62.6 | 62.9 |
| Microbial stearin | 22.4 | 22.4 |
| Microbial olein | 80.9 | 81.5 |

### Visual Effects of Fractionation on Exemplary Microbial Oils of the Disclosure

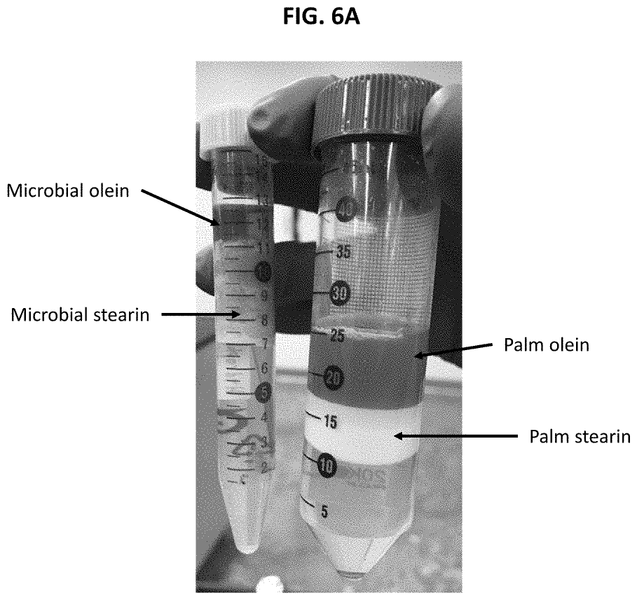

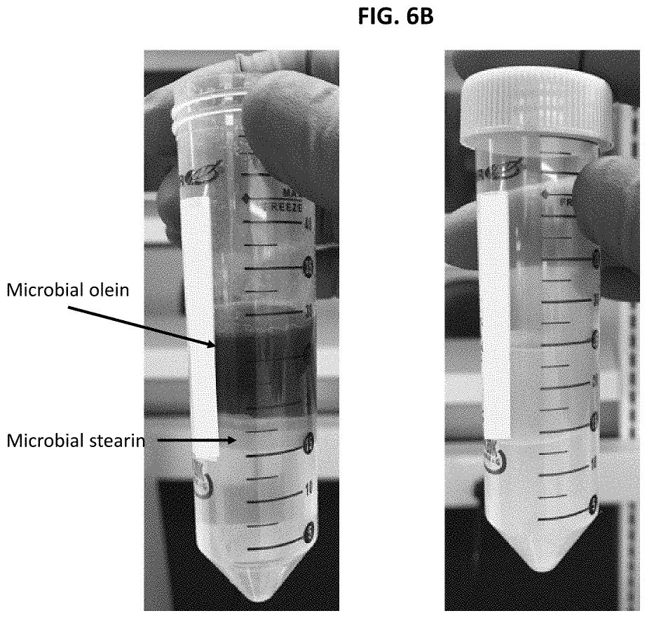

Exemplary crude microbial oils from R. toruloides were fractionated. FIG. 6A-6B exhibit the visual effects of fractionation on various samples. FIG. 6A shows a fractionated microbial oil (left) compared to a fractionated crude palm oil (right). Both fractionated samples contain a top olein layer that is liquid at room temperature and a bottom stearin layer that is solid at room temperature. FIG. 6B shows another fractionated microbial oil (left) and a microbial oil that did not fractionate (right). These images demonstrate a characteristic of exemplary microbial oils of the disclosure which demonstrate the ability to fractionate similar to plant-derived palm oil, a characteristic which does not hold for all microbial oils.

### Example 6

### Sterol Analysis of Exemplary Microbial Oil of the Disclosure

### Materials and Methods

The following procedure was followed in order to measure the content of sterols present in each of these samples: an exemplary microbial oil of the disclosure obtained from R. toruloides (“yeast microbial oil”), Crude Palm Oil (CPO), RBD Palm Oil (RBDPO) and Algae oil. First, each oil was weighed to obtain 40 mg. All oil samples were dissolved in 200 μL of hexane containing 200 μg/mL of a tridecanoic acid methyl ester internal standard (ISTD). The oil samples were then set at 60° C. for 2 h in the vacuum oven to remove the organic solvent by evaporation. Then, one half of each sample was resuspended in 100 μL of pyridine (“plain” preparation). The other half of each sample was resuspended in 100 μL pyridine solution comprising 0.4 mg/mL of each of 5 purified sterol standards corresponding to targets of interest (“spike-in” preparations). Finally, both plain and spike-in preparations were further derivatized by addition of 100 μL of BSTFA+10% TCMS (Thermo Scientific, USA) and incubated at 92° C. for 2 h.

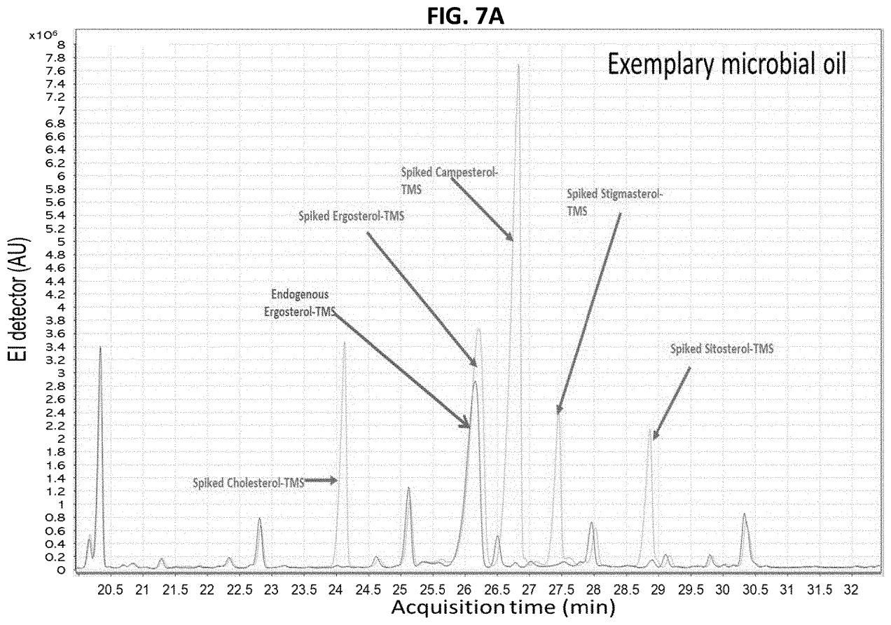

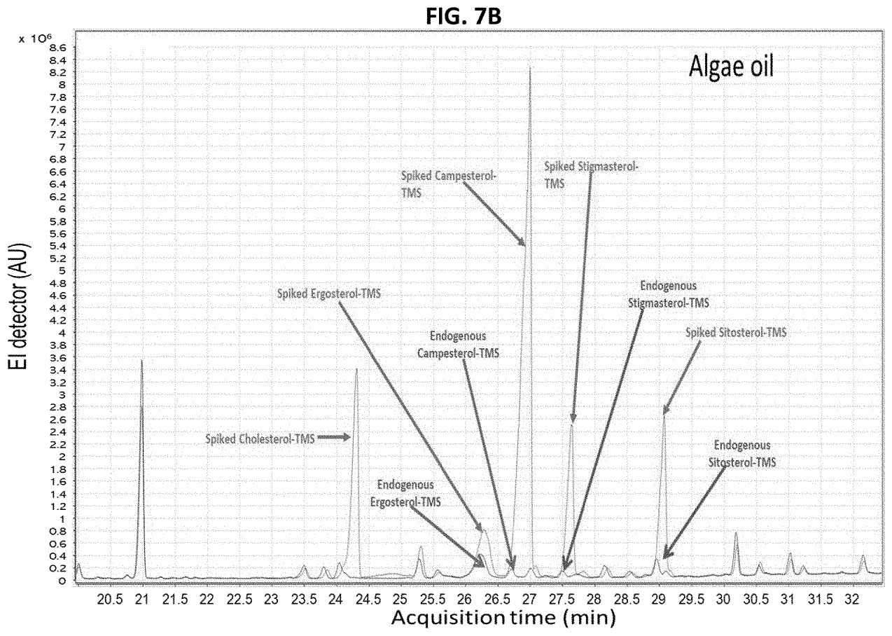

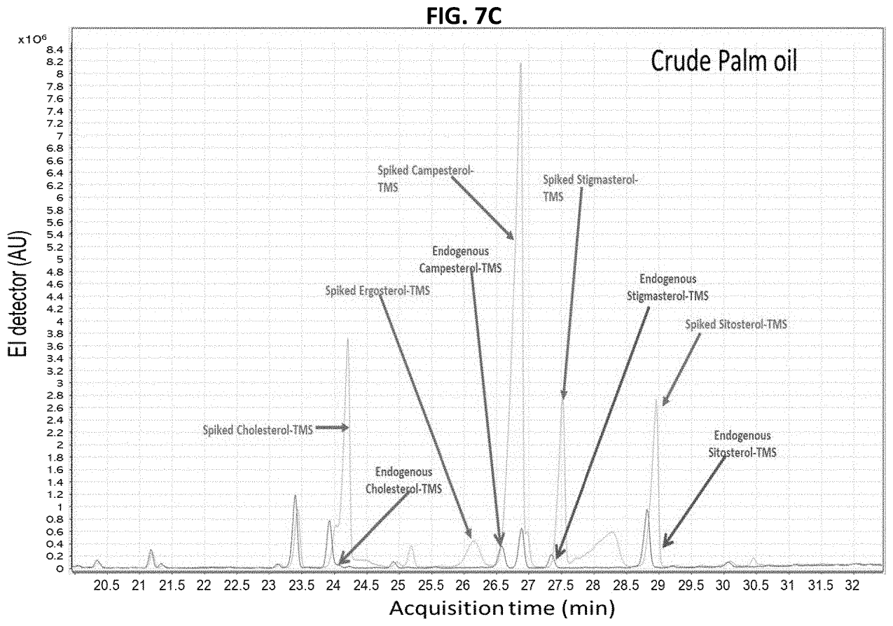

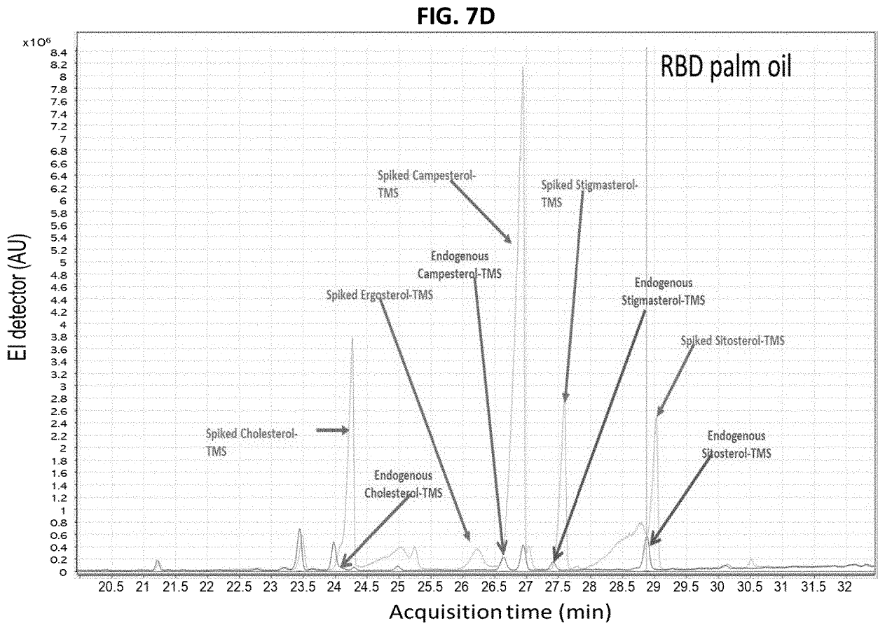

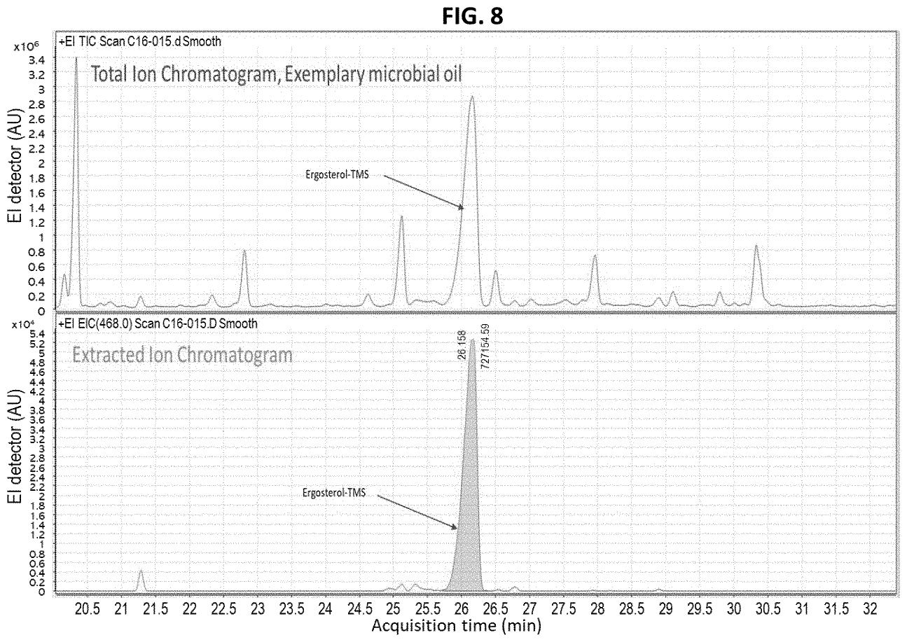

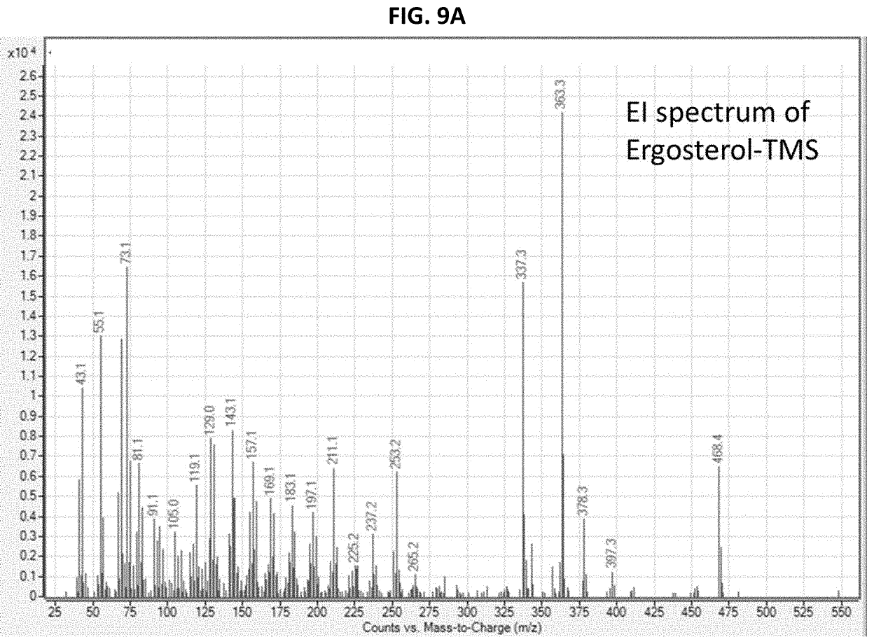

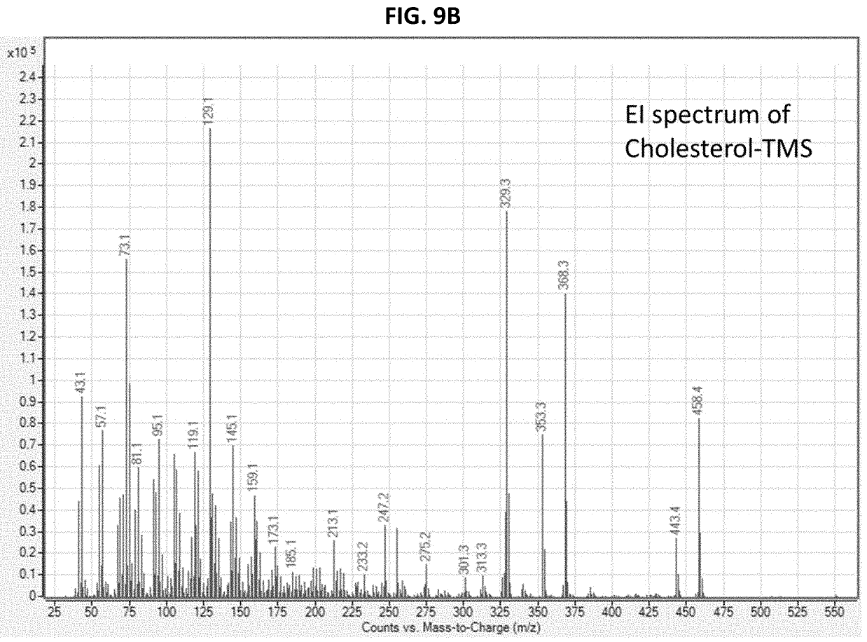

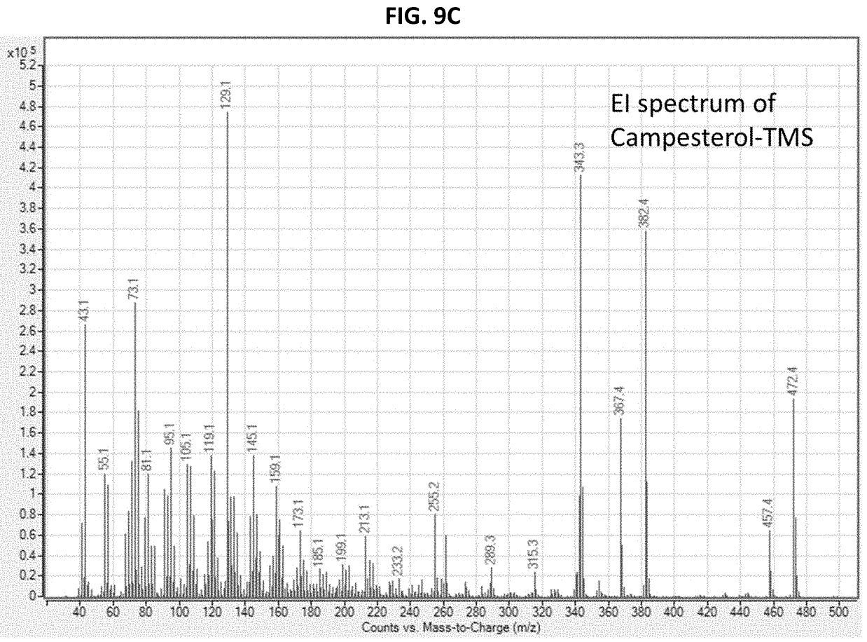

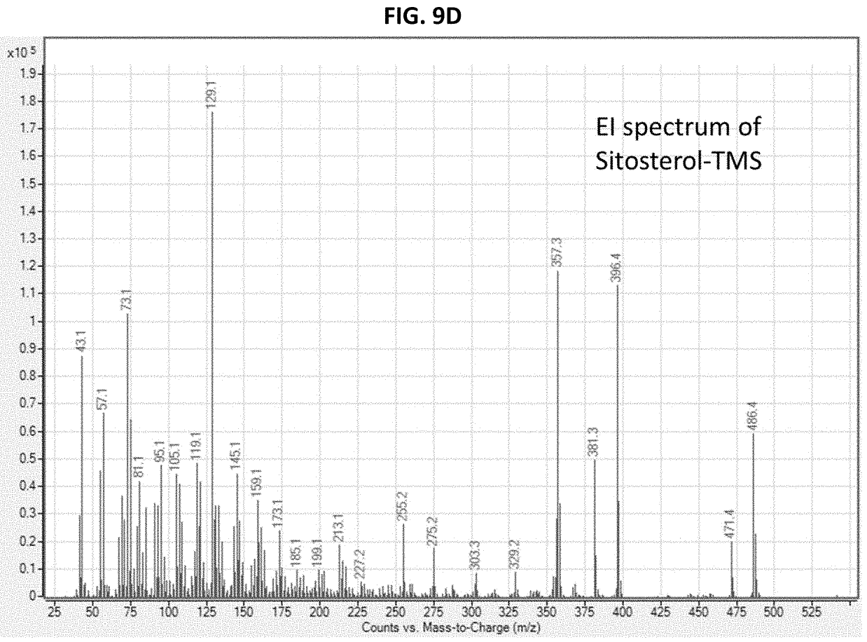

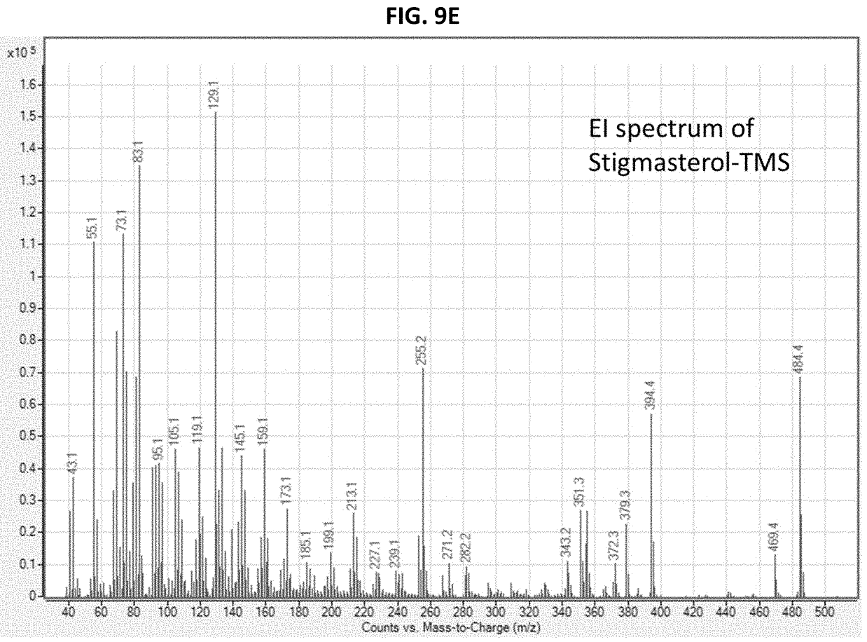

Derivatized oil samples were analyzed using an Agilent® 7890B GC System coupled to an Agilent® 5975 mass selective detector. The GC was operated in splitless mode with constant helium gas flow at 1 mL/min. 1 μL of derivatized oil was injected with the PAL3 Sampler (Model Pal RSI 120 from CTC Analytics, Switzerland) onto an HP-5 ms Ultra Inert column. The total ion chromatograms for each oil (FIG. 7A-7D) were obtained by using a GC oven program as follows: the initial oven temperature was first held at 70° C. for one minute, and then ramped from 70° C. to 255° C. at a rate of 20° C./min; the oven temperature was then further increased at a rate of 1.5° C./min to reach 283° C.; finally, the ramp rate was increased to 15° C./min until the oven temperature reached 300° C., where it was held for 9 min. The total run time was 39 minutes. Peaks representing compounds of interest were extracted and integrated using MassHunter software (Agilent Technologies®, USA), e.g., as visually represented in FIG. 8. Each extracted, integrated peak was then normalized to both the ISTD and their corresponding spike-in sterol peak area. The masses of molecular ions used for extraction are shown in Table 12. All peaks were manually inspected and their electron ionization (EI) spectra were verified relative to known spectra for each sterol. FIG. 9A-9E show illustrative EI spectra for sterols extracted from the crude palm oil spike-in preparation.

TABLE 12
Mass of sterol compounds used for extraction.

| | Molecular |
|---|---|
| Sterol Compounds | Ion (m/z) |
| Cholesterol | 458 |
| Ergosterol | 468 |
| Campesterol | 472 |
| Stigmasterol | 484 |
| Sitosterol | 486 |
| Tridecanoic acid | 228 |
| methyl ester (ISTD) | |

Extracted peaks were first normalized to the ISTD peak for the corresponding runs. For each spike-in run, residual peaks for each sterol standard were calibrated by subtracting normalized peak areas of the plain runs from the spike-in runs. Residual peaks for each sterol were averaged across the 4 oil sample runs, and then used to re-normalize plain peak areas for differences in detector signal across targets. These final, re-normalized peak areas were used to calculate total sterol content (Table 13) and sterol profiles (Table 14) for each of the oil samples.

TABLE 13
Total sterol content.

| | Total sterols |
|---|---|
| Sample | (ppm) |
| Yeast microbial oil | 2297 |
| Crude palm oil | 452 |
| RBD palm oil | 251 |
| Algae oil | 388 |

TABLE 14
Sterol profiles.

| | Yeasst | Crude | RBD | |
|---|---|---|---|---|
| | microbial | palm | palm | Algae |
| Sterol | oil | oil | oil | oil |
| Ergosterol | 100% | n.d. | n.d. | 50.81% |
| Cholesterol | n.d. | 1.71% | 1.58% | n.d. |
| Campesterol | n.d. | 5.49% | 5.20% | 2.75% |
| Stigmasterol | n.d. | 14.57% | 15.82% | 12.80% |
| Sitosterol | n.d. | 78.22% | 77.39% | 33.63% |

The results demonstrate that an exemplary yeast microbial oil of the disclosure only comprised ergosterol and did not comprise cholesterol, campesterol, stigmasterol, or sitosterol, in contrast to the other three samples derived from agricultural palm plants or algae.

### Example 7

### Carotenoid Analysis of Exemplary Microbial Oils of the Disclosure

### Oil Samples

Six oil samples were analyzed to identify the carotenoids present within each one.

Sample 1: agricultural palm oil.

Sample 2: exemplary microbial oil of the disclosure obtained from R. toruloides; strong acid (H₂SO₄) treatment with solvent extraction of lipids.

Sample 3: exemplary microbial oil of the disclosure obtained from R. toruloides; strong acid (HCl) treatment with solvent extraction of lipids.

Sample 4: exemplary microbial oil of the disclosure obtained from R. toruloides; weak acid (H₃PO₄) treatment with solvent extraction of lipids.

Sample 5: exemplary microbial oil of the disclosure obtained from R. toruloides; acid-free extraction of lipids.

Sample 6: exemplary microbial oil of the disclosure obtained from R. toruloides; acid-free extraction of lipids.

### Carotenoid Analysis Materials and Methods

Sample Preparation. Oil samples were diluted in diethyl ether. Each solution was saponified in homogeneous phase for 1 hr. After acidification and washing, UV/Vis and HPLC analysis were performed.

UV/Vis analysis. For each sample, an initial overall UV/Vis absorbance spectrum was collected between 200 and 600 nm wavelengths. This overall spectrum shows the total overlapping absorbance of all of the sample's carotenoids, which allows for estimation of the total carotenoid content within the sample. UV/Vis spectra were recorded with a Jasco V-530 spectrophotometer in benzene. (E^{1%}/1 cm=2500)

High performance liquid chromatography (HPLC) diode array detector (DAD) analysis. The HPLC-DAD assay was conducted using a Dionex Ultimate 3000 HPLC system detecting absorbance at λ=450 nm. Temperature was maintained at 22° C. Data acquisition was performed by Chromeleon 7.2 software. The column employed was a YMC Carotenoid C30 column, with 3 μM bead size and dimensions of 250×4.6 mm i.d. Buffer A had the following composition: 81% MeOH, 15% TBME, 4% H₂O. Buffer B had this composition: 6% MeOH, 90% TBME, 4% H₂O. The chromatograms were performed in linear gradient: 0 min 100% Buffer A to 70 min 70% Buffer B. The flow rate was maintained at 1.00 cm³/min.

Carotenoid identification. An absorbance spectrum was collected for each analyte with a corresponding peak in the HPLC-DAD chromatogram. Identities of individual carotenoids were confirmed based on comparing the retention time and UV/Vis spectrum for that analyte to known standards.

### Results

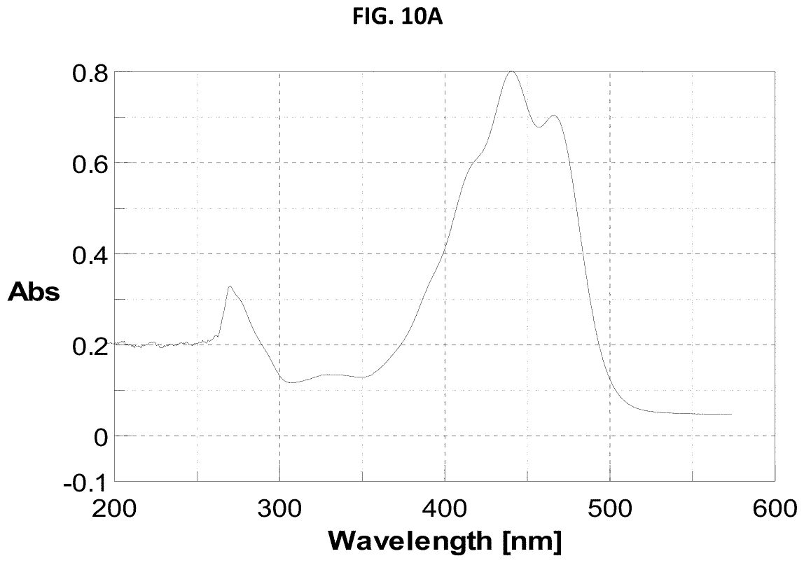

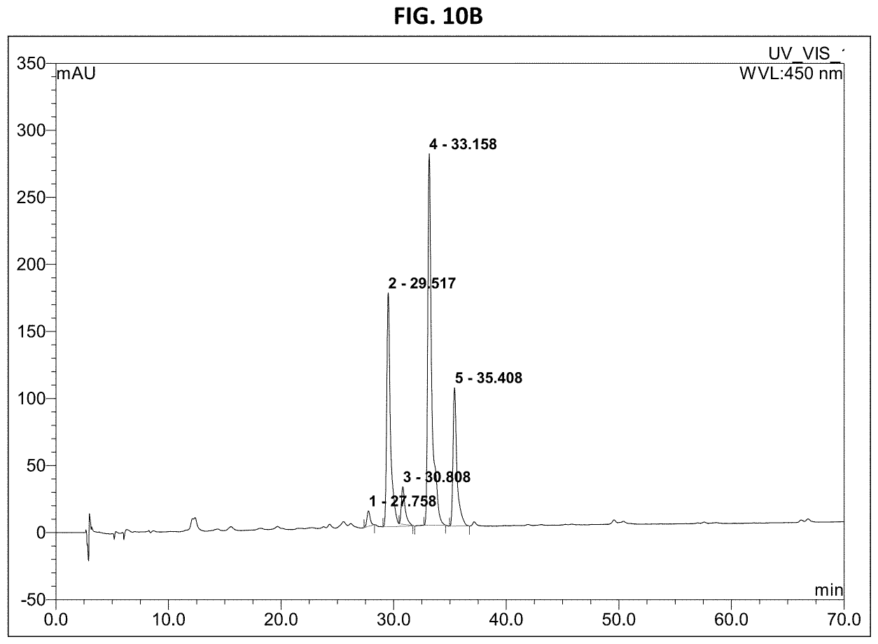

Sample 1. The overall UV/Vis absorbance spectrum for Sample 1, agricultural palm oil, is shown in FIG. 10A with the absorbance at individual wavelengths identified in Table 15. The overall UV/Vis spectrum shows the expected distribution centered around 450 nm. The total carotenoid content, roughly estimated using the absorbance at 459 nm, was determined to be approximately 478 ppm.

TABLE 15
Sample 1, UV/Vis Abs at specific wavelengths.

| Peak # | λ (nm) | Abs |
|---|---|---|
| 1 | 279 | 0.29772 |
| 2 | 433 | 0.58054 |
| 3 | 459 | 0.7978 |
| 4 | 486 | 0.69501 |

For Sample 1, the HPLC-DAD chromatogram reporting absorbance at 450 nm is shown in FIG. 10B with individual peaks identified in Table 16. As expected, this sample contained the known agricultural palm oil-associated carotenoids α- and β-carotene, and derivatives thereof.

TABLE 16
Sample 1, HPLC peak identification.

| Peak | Ret. Time | | Height | Area | Rel.Area | |
|---|---|---|---|---|---|---|
| No. | (min) | Peak Name | (mAU) | mAU*min | (%) | Type |
| 1 | 27.76 | (13Z)-β-Carotene | 11.517 | 3.793 | 1.59 | BMB |
| 2 | 29.52 | α-Carotene | 174.265 | 68.511 | 28.75 | BMB |
| 3 | 30.81 | (13Z)-α-Carotene | 27.930 | 10.790 | 4.53 | Rd |
| 4 | 33.16 | β-Carotene | 277.067 | 113.661 | 47.69 | BMB |
| 5 | 35.41 | (9Z)-β-Carotene. | 103.203 | 41.585 | 17.45 | BMB |
| Total: | | | 593.982 | 238.341 | 100.00 | |

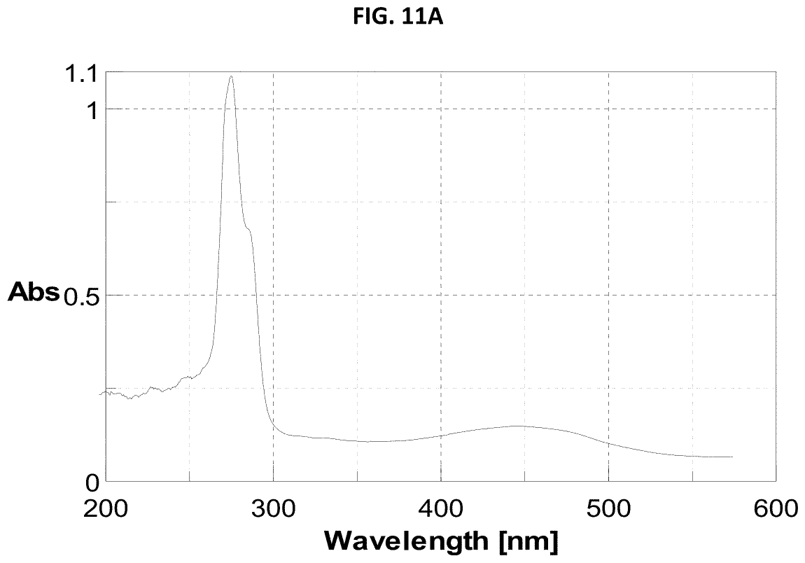

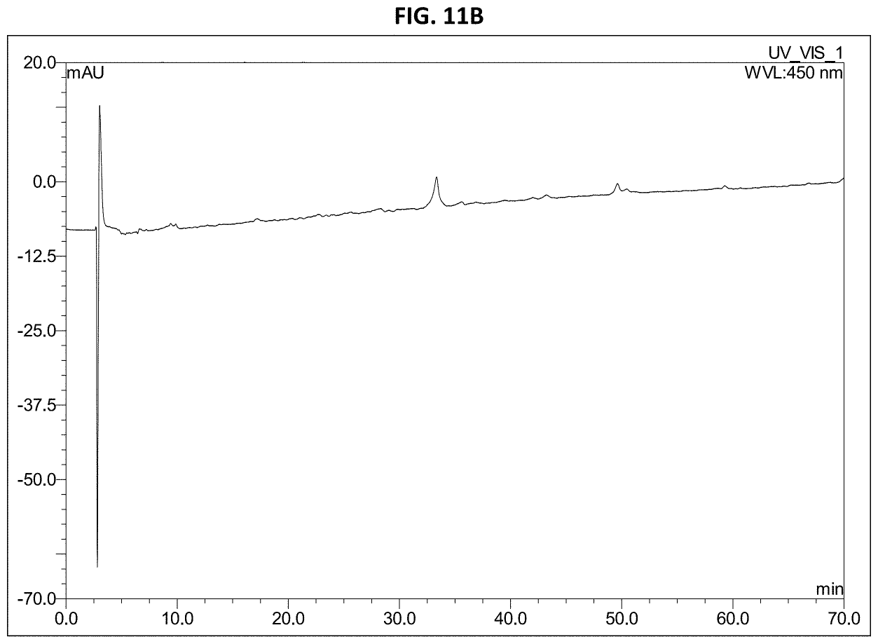

Sample 2. The overall UV/Vis absorbance spectrum for Sample 2, strong acid-extracted microbial oil, is shown in FIG. 11A. The overall UV/Vis spectrum shows essentially no absorbance in the 300-500 nm range, likely because of carotenoid degradation due to the strong acid treatment. For Sample 2, the HPLC-DAD chromatogram reporting absorbance at 450 nm is shown in FIG. 11B with no identifiable peaks.

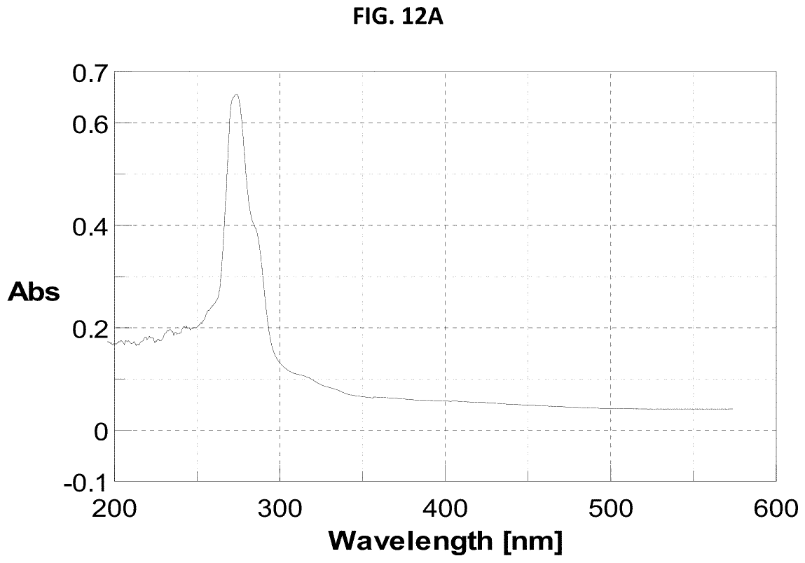

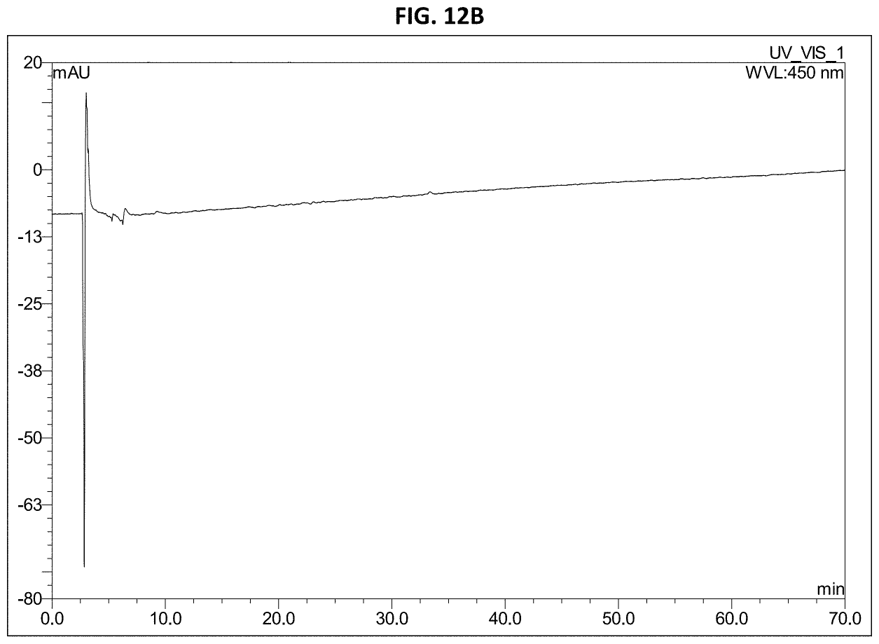

Sample 3. The overall UV/Vis absorbance spectrum for Sample 3, strong acid-extracted microbial oil, is shown in FIG. 12A. The overall UV/Vis spectrum shows essentially no absorbance in the 300-500 nm range, likely because of carotenoid degradation due to the strong acid treatment. For Sample 3, the HPLC-DAD chromatogram reporting absorbance at 450 nm is shown in FIG. 12B with no identifiable peaks.

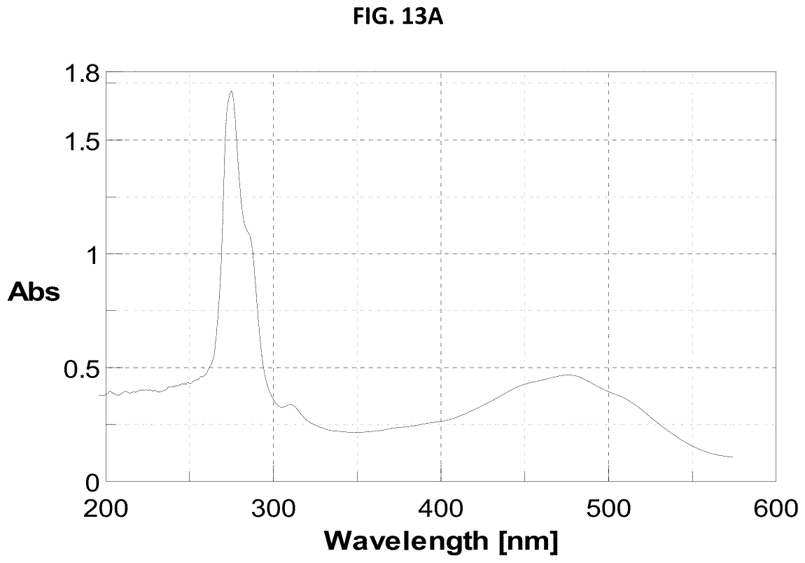

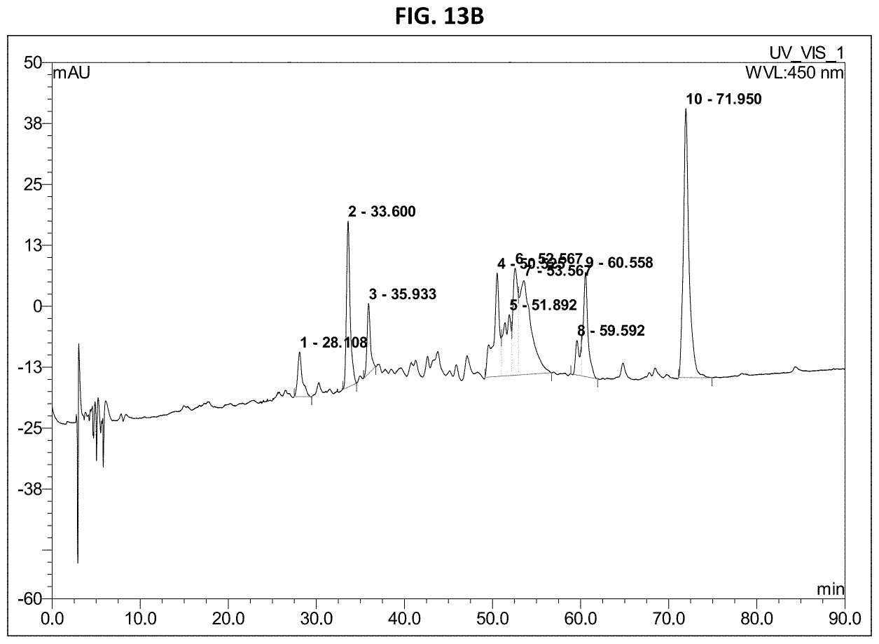

Sample 4. The overall UV/Vis absorbance spectrum for Sample 4, weak acid-extracted microbial oil, is shown in FIG. 13A. The total carotenoid content, roughly estimated using the absorption at 496 nm, was determined to be approximately 169 ppm. For Sample 4, the HPLC-DAD chromatogram reporting absorbance at 450 nm is shown in FIG. 13B with individual peaks identified in Table 17. As expected for a microbial oil from R. toruloides, the microbial oil was identified as comprising both torularhodin and torulene, as well as other unidentified carotenoids some of which may correspond to derivatives of these carotenoids. The sample also contained β-carotene and derivatives thereof.

TABLE 17
Sample 4, HPLC peak identification.

| Peak | Ret. Time | | | Area | Rel.Area | |
|---|---|---|---|---|---|---|
| No. | (min) | Peak Name | λₘₐₓ (nm) | mAU*min | (%) | Type |
| 1 | 28.11 | (13Z)-β-Carotene | 443, 469 | 5.562 | 3.52 | BMB* |
| 2 | 33.60 | β-Carotene | 451, 477 | 16.376 | 10.35 | BMB |
| 3 | 35.93 | (9Z)-β-Carotene | 446, 471 | 6.326 | 4.00 | BMB |
| 4 | 50.53 | Unidentified (ui) | 384, 464, 488 | 16.446 | 10.40 | BM* |
| 5 | 51.89 | ui | 382, 473 | 11.777 | 7.45 | M* |
| 6 | 52.57 | Torularhodin | 496, 527 | 13.675 | 8.65 | M* |
| 7 | 53.57 | ui | 447, 473, 503 | 29.848 | 18.87 | MB* |
| 8 | 59.59 | ui. | not detected | 3.951 | 2.50 | BM* |
| 9 | 60.56 | ui | 457, 482, 514 | 13.147 | 8.31 | MB* |
| 10 | 71.95 | Torulene | 461, 486, 519 | 41.050 | 25.96 | BMB* |
| Total: | | | | 158.157 | 100.00 | |

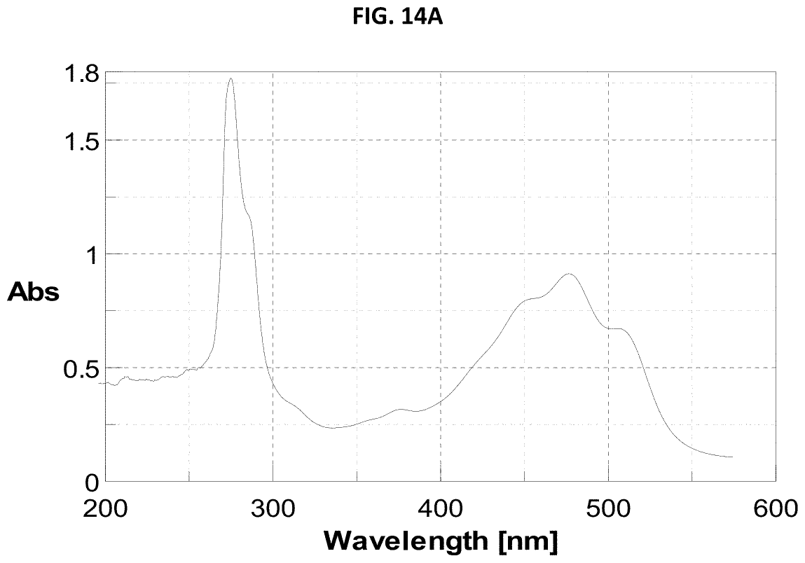

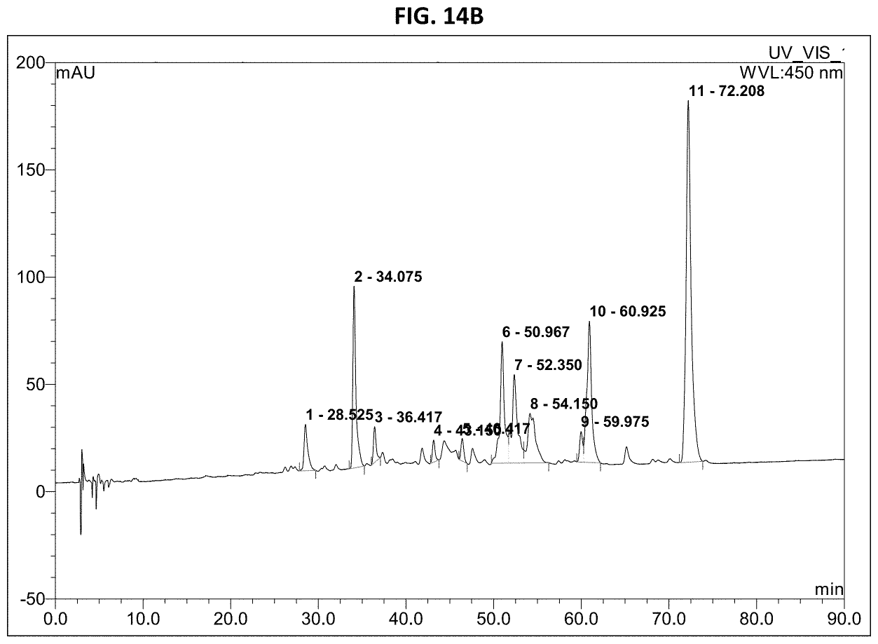

Sample 5. The overall UV/Vis absorbance spectrum for Sample 5, acid-free extracted microbial oil, is shown in FIG. 14A with the absorbance at individual wavelengths identified in Table 18. The overall UV/Vis spectrum shows a peak around 475 nm. The total carotenoid content, roughly estimated using the absorbance at 496 nm, was determined to be approximately 471 ppm.

TABLE 18
Sample 5, UV/Vis Abs at specific wavelengths.

| Peak # | λ (nm) | Abs |
|---|---|---|
| 1 | 283 | 1.76214 |
| 2 | 470 | 0.73005 |
| 3 | 496 | 0.85332 |
| 4 | 529 | 0.59645 |

For Sample 5, the HPLC-DAD chromatogram reporting absorbance at 450 nm is shown in FIG. 14B with individual peaks identified in Table 19. As with sample 4, this sample contained torulene, possible derivatives of torulene, β-carotene and β-carotene derivatives.

TABLE 19
Sample 5, HPLC peak identification.

| Peak | Ret. Time | | | Area | Rel.Area | |
|---|---|---|---|---|---|---|
| No. | (min) | Peak Name | λₘₐₓ (nm) | mAU*min | (%) | Type |
| 1 | 28.53 | (13Z)-β-Carotene | 443, 469 | 10.770 | 3.47 | BMB |
| 2 | 34.08 | β-Carotene | 451, 477 | 34.796 | 11.20 | BMB |
| 3 | 36.42 | (9Z)-β-Carotene | 446, 471 | 5.851 | 1.88 | BMB |
| 4 | 43.15 | unidentified | 434, 456, 484, | 3.528 | 1.14 | BMB |
| 5 | 46.42 | ui | not detected | 3.873 | 1.25 | BMB |
| 6 | 50.97 | ui Z-isomer | 381, 480 | 34.250 | 11.03 | BM* |
| 7 | 52.35 | ui | 452, 479, 511 | 30.971 | 9.97 | M* |
| 8 | 54.15 | ui. | 449, 473, 503 | 24.712 | 7.96 | MB* |
| 9 | 59.98 | ui | 452, 477, 508 | 5.799 | 1.87 | BM* |
| 10 | 60.93 | ui | 457, 482, 514 | 42.164 | 13.58 | MB* |
| 11 | 72.21 | Torulene | 461, 486, 519 | 113.867 | 36.66 | BMB |
| Total: | | | | 310.583 | 100.00 | |

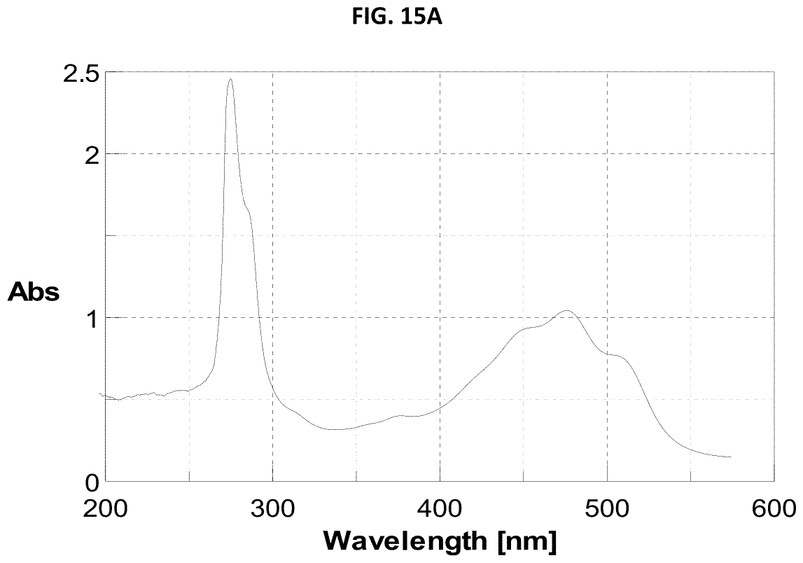

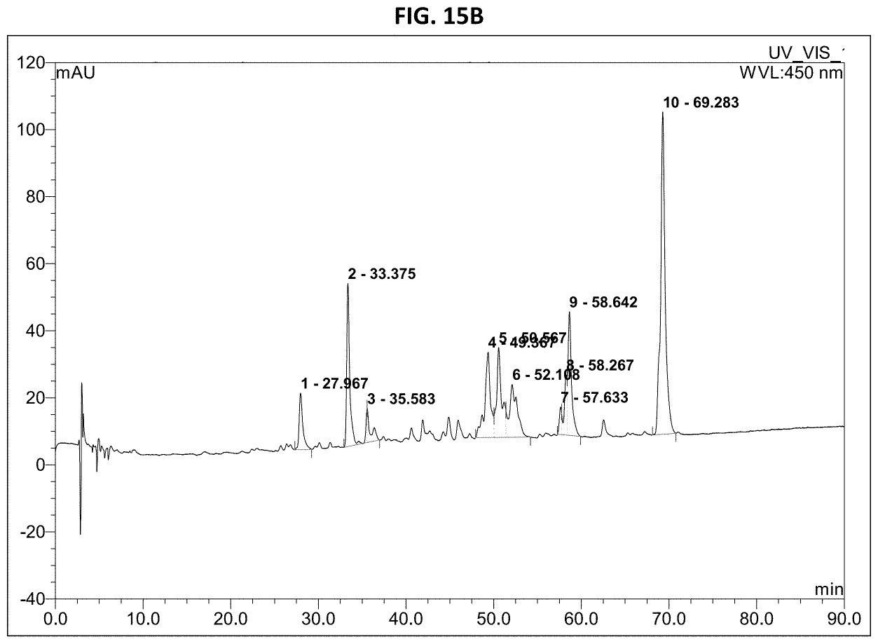

Sample 6. The overall UV/Vis absorbance spectrum for Sample 6, acid-free extracted microbial oil, is shown in FIG. 15A with the absorbance at individual wavelengths identified in Table 20. The overall UV/Vis spectrum shows a peak around 475 nm. The total carotenoid content, roughly estimated using the absorbance at 496 nm, was determined to be approximately 802 ppm.

TABLE 20
Sample 6, UV/Vis Abs at specific wavelengths.

| Peak # | λ (nm) | Abs |
|---|---|---|
| 1 | 283 | 2.44332 |
| 2 | 467 | 0.81861 |
| 3 | 496 | 0.94825 |
| 4 | 529 | 0.65319 |

For Sample 6, the HPLC-DAD chromatogram reporting absorbance at 450 nm is shown in FIG. 15B with individual peaks identified in Table 21. As with samples 4 and 5, this sample contained torulene, possible derivatives of torulene, β-carotene and β-carotene derivatives.

TABLE 21
Sample 6, HPLC peak identification.

| Peak | Ret. Time | | | Area | Rel.Area | |
|---|---|---|---|---|---|---|
| No. | (min) | Peak Name | λₘₐₓ (nm) | mAU*min | (%) | Type |
| 1 | 27.97 | (13Z)-β-Carotene | 443, 469 | 8.173 | 4.74 | BMB |
| 2 | 33.38 | β-Carotene | 451, 477 | 20.985 | 12.18 | BM* |
| 3 | 35.58 | (9Z)-β-Carotene | 446, 471 | 4.204 | 2.44 | MB* |
| 4 | 49.37 | ui. mixture | 384, 464, 488 | 19.266 | 11.18 | BM* |
| 5 | 50.57 | unidentified | 452, 479, 511 | 17.971 | 10.43 | M* |
| 6 | 52.11 | ui | 447, 473, 503 | 16.588 | 9.63 | MB* |
| 7 | 57.63 | ui | not detected | 2.188 | 1.27 | BM * |
| 8 | 58.27 | ui | nd | 6.683 | 3.88 | M* |
| 9 | 58.64 | ui. | 457, 482, 514 | 17.293 | 10.04 | MB* |
| 10 | 69.28 | Torulene | 461, 486, 519 | 58.958 | 34.22 | BMB |
| Total: | | | | 172.311 | 100.00 | |

Overall, these results demonstrate that exemplary microbial oils of the disclosure comprise torulenes and/or torulorhodins, as well as β-carotene and derivatives thereof. This is in contrast to agricultural palm oil, which contains predominantly α- and β-carotenes and derivatives thereof.

### NUMBERED EMBODIMENTS OF THE INVENTION

Notwithstanding the appended claims, the disclosure sets forth the following numbered embodiments:
1. A refined, bleached, and/or deodorized (RBD) microbial oil composition produced by an oleaginous yeast.
2. A refined, bleached, and/or deodorized (RBD) microbial oil composition produced by an oleaginous yeast, wherein the composition comprises ergosterol and does not comprise campesterol, β-sitosterol, or stigmasterol.
3. A refined and/or deodorized microbial oil composition produced by an oleaginous yeast, wherein the composition comprises at least one pigment selected from the group consisting of carotene, torulene and torulorhodin and does not comprise chlorophyll.
4. The composition of embodiment 3, wherein the composition is bleached, thereby producing an RBD microbial oil composition, but wherein a measurable amount of the pigment remains.
5. A refined, bleached, and/or deodorized (RBD) microbial oil composition produced by an oleaginous yeast, wherein the composition is fractionable into two fractions, wherein the two fractions are microbial olein and microbial stearin, wherein each fraction comprises at least 10% of the composition's original mass, and wherein the iodine value (IV) of the fractions differs by at least 10.
6. A microbial oil composition produced by an oleaginous yeast, wherein the composition comprises the following amounts of fatty acids relative to the total fatty acids:
   a) at least about 30% w/w saturated fatty acids with chain lengths between 16 and 18 carbons long;
   b) at least about 30% w/w unsaturated fatty acids with 18 carbon chain lengths; and
   c) less than about 30% w/w total polyunsaturated fatty acids.
7. A refined, bleached, and/or deodorized (RBD) microbial oil composition produced by an oleaginous yeast, wherein the composition has one or more characteristics similar to plant-derived palm oil selected from the group consisting of: apparent density, refractive index, saponification value, unsaponifiable matter, iodine value, slip melting point, fatty acid composition, triglyceride content, overall saturation level, and level of mono- and poly-unsaturated fatty acids.
8. A microbial oil composition produced by an oleaginous yeast, comprising:
   a) at least about 30% w/w saturated fatty acids with chain lengths between 16 and 18 carbons long;
   b) at least about 30% w/w unsaturated fatty acids with 18 carbon chain lengths;
   c) less than about 30% w/w total polyunsaturated fatty acids;
   d) at least about 50 ppm ergosterol;
   wherein the composition does not contain a phytosterol or chlorophyll, and wherein the composition has one or more characteristics similar to plant-derived palm oil selected from the group consisting of iodine value, triglyceride content, slip melting point, oxidative stability, and overall saturation level.
9. The composition of any one of embodiments 1-8, wherein the composition comprises 10-45% C16 saturated fatty acid.
10. The composition of any one of embodiments 1-9, wherein the composition comprises 10-70% C18 unsaturated fatty acid.
11. The composition of any one of embodiments 1-10, wherein the composition comprises 3-30% C18 saturated fatty acid.
12. The composition of any one of embodiments 1-11, wherein the composition comprises a saponification value similar to that of plant-derived palm oil.
13. The composition of any one of embodiments 1-12, wherein the composition comprises a saponification value of 150-210.
14. The composition of any one of embodiments 1-13, wherein the composition comprises an iodine value similar to that of plant-derived palm oil.
15. The composition of any one of embodiments 1-14, wherein the composition comprises an iodine value of 50-65.
16. The composition of any one of embodiments 1-15, wherein the composition comprises a slip melting point similar to that of plant-derived palm oil.
17. The composition of any one of embodiments 1-16, wherein the composition comprises a slip melting point of 30° C.-40° C.
18. The composition of any one of embodiments 1-17, wherein the composition comprises a saturated fatty acid composition similar to that of plant-derived palm oil.
19. The composition of any one of embodiments 1-18, wherein the composition comprises a saturated fatty acid composition of at least 30%.
20. The composition of any one of embodiments 1-19, wherein the composition comprises a saturated fatty acid composition of at most 70%.
21. The composition of any one of embodiments 1-20, wherein the composition comprises an unsaturated fatty acid composition similar to that of plant-derived palm oil.
22. The composition of any one of embodiments 1-21, wherein the composition comprises an unsaturated fatty acid composition of at least 30%.
23. The composition of any one of embodiments 1-22, wherein the composition comprises an unsaturated fatty acid composition of at most 70%.
24. The composition of any one of embodiments 1-23, wherein the composition comprises a mono- and poly-unsaturated fatty acid composition similar to that of plant-derived palm oil.
25. The composition of any one of embodiments 1-24, wherein the composition comprises 30-50% mono-unsaturated fatty acids as a percentage of overall fatty acids.
26. The composition of any one of embodiments 1-25, wherein the composition comprises 5-25% poly-unsaturated fatty acids as a percentage of overall fatty acids.
27. The composition of any one of embodiments 1-26, wherein the composition comprises a triglyceride content similar to that of plant-derived palm oil.
28. The composition of any one of embodiments 1-27, wherein the composition comprises a triglyceride content of 90-98% as a percentage of overall glycerides.
29. The composition of any one of embodiments 1-28, wherein the composition comprises less than 100 ppm of, comprises less than 50 ppm of, or does not comprise a sterol selected from a phytosterol, cholesterol, or a protothecasterol.
30. The composition of any one of embodiments 1-29, wherein the composition comprises less than 100 ppm of, comprises less than 50 ppm of, or does not comprise a phytosterol.
31. The composition of any one of embodiments 1-30, wherein the composition comprises less than 100 ppm of, comprises less than 50 ppm of, or does not comprise a phytosterol selected from the group consisting of campesterol, β-sitosterol, stigmasterol.
32. The composition of any one of embodiments 1-31, wherein the composition comprises less than 100 ppm of, comprises less than 50 ppm of, or does not comprise cholesterol.
33. The composition of any one of embodiments 1-32, wherein the composition comprises less than 100 ppm of, comprises less than 50 ppm of, or does not comprise protothecasterol.
34. The composition of any one of embodiments 1-33, wherein the composition comprises ergosterol, comprises at least 50 ppm ergosterol, or comprises at least 100 ppm ergosterol.
35. The composition of any one of embodiment 1-34, wherein the composition comprises an ergosterol content of at least 60% w/w as a percentage of overall sterols.
36. The composition of any one of embodiments 1-35, wherein the composition does not comprise a pigment.
37. The composition of any one of embodiments 1-36, wherein the composition does not comprise chlorophyll.
38. The composition of any one of embodiments 1-37, wherein the composition comprises a pigment selected from the group consisting of carotene, torulene and torulorhodin.
39. The composition of any one of embodiments 1-38, wherein the composition comprises each of carotene, torulene and torulorhodin.
40. The composition of any one of embodiments 1-39, wherein the composition comprises at least 10 ppm, at least 50 ppm, or at least 100 ppm carotene.
41. The composition of any one of embodiments 1-40, wherein the composition comprises carotene, and wherein the carotene is β-carotene and/or a derivative thereof.
42. The composition of any one of embodiments 1-41, wherein the composition comprises at least 10 ppm, at least 50 ppm, or at least 100 ppm torulene and/or a derivative thereof.
43. The composition of any one of embodiments 1-42, wherein the composition comprises at least 10 ppm, at least 50 ppm, or at least 100 ppm torulorhodin and/or a derivative thereof.
44. The composition of any one of embodiments 1-43, wherein the oleaginous yeast is a recombinant yeast.
45. The composition of any one of embodiments 1-44, wherein the oleaginous yeast is of the genus Yarrowia, Candida, Rhodotorula, Rhodosporidium, Metschnikowia, Cryptococcus, Trichosporon, or Lipomyces.
46. The composition of any one of embodiments 1-45, wherein the oleaginous yeast is of the genus Rhodosporidium.
47. The composition of any one of embodiments 1-46, wherein the oleaginous yeast is of the species Rhodosporidium toruloides.
48. The composition of any one of embodiments 1-47, wherein the composition is fractionable.
49. The composition of any one of embodiments 1-48, wherein the composition may be fractionated into microbial olein and microbial stearin.
50. The composition of any one of embodiments 1-49, wherein the composition may be fractionated into microbial olein and microbial stearin, and wherein each fraction comprises at least 10% of the composition's starting mass.
51. The composition of any one of embodiments 1-50, wherein the composition may be fractionated into microbial olein and microbial stearin, and wherein the iodine value (IV) of the fractions differs by at least 10.
52. The composition of any one of embodiments 1-51, wherein the composition may be fractionated into microbial olein and microbial stearin, and wherein the IV of the fractions differs by at least 20.
53. The composition of any one of embodiments 1-52, wherein the composition may be fractionated into microbial olein and microbial stearin, and wherein the IV of the fractions differs by at least 30.
54. A microbial oil composition produced by an oleaginous yeast, wherein the composition comprises:
   a) less than 10% w/w palmitic-palmitic-palmitic triglycerides;
   b) greater than 15% w/w palmitic-palmitic-oleic triglycerides; and
   c) greater than 15% w/w oleic-oleic-palmitic triglycerides.
55. The microbial oil composition of embodiment 54, wherein said palmitic-palmitic-palmitic triglyceride content is between about 0.8% and 1.3% w/w.
56. The microbial oil composition of any one of embodiments 54-55, wherein said palmitic-palmitic-oleic triglyceride content is between about 16.9% and 28.2% w/w.
57. The microbial oil composition of any one of embodiments 54-56, wherein said oleic-oleic-palmitic triglyceride content is between about 15.7% and 26.0% w/w.
58. The microbial oil composition of any one of embodiments 54-57, further comprising a stearic-stearic-oleic triglyceride content of less than 10% w/w and a stearic-oleic-oleic triglyceride content of less than 10% w/w.
59. The microbial oil composition of any one of embodiments 54-58, wherein said stearic-stearic-oleic triglyceride content is between about 1.2% and 1.9% w/w.
60. The microbial oil composition of any one of embodiments 54-59, wherein said stearic-oleic-oleic triglyceride content is between about 3.2% and 5.4% w/w.
61. A microbial oil composition produced by an oleaginous yeast, wherein the composition comprises triglycerides, and wherein greater than 40% of said triglycerides have one unsaturated sidechain.
62. The microbial oil composition of embodiment 61, wherein greater than 30% of said triglycerides have two unsaturated sidechains.
63. The composition of any one of embodiments 54-62, wherein between 10% and 15% of palmitic and/or stearic fatty acids are located at the sn-2 position of triglyceride molecules.
64. A microbial oil composition produced by an oleaginous yeast, wherein the composition comprises the following amounts of fatty acids relative to the total fatty acids:
   a) between about 7.0% and 35% stearic acid;
   b) between about 10% and 50% oleic acid; and
   c) between about 8% and 20% linoleic acid.
65. The composition of any one of embodiments 1-64, wherein the composition further comprises a feedstock as recited in International Patent Application No. PCT/US2021/015302.
66. The composition of any one of embodiments 1-65, wherein the composition is produced via a method recited in International Patent Application No. PCT/US2021/015302.
67. A method of producing a microbial oil composition according to any one of embodiments 1-66, the method comprising the steps of:
   a) providing an oleaginous yeast and a carbon source, and
   b) culturing said oleaginous yeast, thereby producing said microbial oil.
68. The method of embodiment 67, further comprising a composition or method step disclosed in International Patent Application No. PCT/US2021/015302.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not, be taken as an acknowledgement or any form of suggestion that they constitute valid prior art or form part of the common general knowledge in any country in the world. The following international PCT application is incorporated herein by reference in its entirety: International Patent Application No. PCT/US2021/015302.

1. A refined, bleached, and/or deodorized (RBD) microbial oil composition produced by an oleaginous yeast.

2-8. (canceled)

9. The composition of claim 1, wherein the composition comprises 10-45% C16 saturated fatty acid.

10. The composition of claim 1, wherein the composition comprises 10-70% C18 unsaturated fatty acid.

11. The composition of claim 1, wherein the composition comprises 3-30% C18 saturated fatty acid.

12-18. (canceled)

19. The composition of claim 1, wherein the composition comprises a saturated fatty acid composition of at least 10%.

20. The composition of claim 1, wherein the composition comprises a saturated fatty acid composition of at most 70%.

21. (canceled)

22. The composition of claim 1, wherein the composition comprises an unsaturated fatty acid composition of at least 30%.

23. The composition of claim 1, wherein the composition comprises an unsaturated fatty acid composition of at most 70%.

24. (canceled)

25. The composition of claim 1, wherein the composition comprises about 5-60% mono-unsaturated fatty acids as a percentage of overall fatty acids.

26. The composition of claim 1, wherein the composition comprises less than about 30% w/w total polyunsaturated fatty acid.

27. (canceled)

28. The composition of claim 1, wherein the composition comprises a triglyceride content of 90-98% as a percentage of overall glycerides.

29. The composition of claim 1, wherein the composition does not comprise a sterol selected from a phytosterol, cholesterol, or a protothecasterol.

30. (canceled)

31. The composition of claim 1, wherein the composition does not comprise a phytosterol selected from the group consisting of campesterol, β-sitosterol, a stigmasterol.

32-33. (canceled)

34. The composition of claim 1, wherein the composition comprises ergosterol, comprises at least 50 ppm ergosterol, or comprises at least 100 ppm ergosterol.

35. The composition of claim 1, wherein the composition comprises an ergosterol content of at least 60% w/w as a percentage of overall sterols.

36. The composition of claim 1, wherein the composition does not comprise a pigment.

37. The composition of claim 1, wherein the composition does not comprise chlorophyll.

38. The composition of claim 1, wherein the composition comprises a pigment selected from the group consisting of carotene, torulene and torulorhodin.

39-43. (canceled)

44. The composition of claim 1, wherein the oleaginous yeast is a recombinant yeast.

45. The composition of claim 1, wherein the oleaginous yeast is of the genus Yarrowia, Candida, Rhodotorula, Rhodosporidium, Metschnikowia, Cryptococcus, Trichosporon, or Lipomyces.

46. The composition of claim 1, wherein the oleaginous yeast is of the genus Rhodosporidium.

47. The composition of claim 1, wherein the oleaginous yeast is of the species Rhodosporidium toruloides.

48. The composition of claim 1, wherein the composition is fractionable.

49. The composition of claim 1, wherein the composition is fractionable and wherein:
a) the composition may be fractionated into microbial olein and microbial stearin;
b) each fraction comprises at least 10% of the composition's starting mass; and/or
c) the iodine value (IV) of the fractions differs by at least 10.

50-53. (canceled)

54. The composition of claim 1, wherein the composition comprises:
a) less than 10% w/w palmitic-palmitic-palmitic triglycerides;
b) greater than 15% w/w palmitic-palmitic-oleic triglycerides; and
c) greater than 15% w/w oleic-oleic-palmitic triglycerides.

55-57. (canceled)

58. The composition of claim 1, wherein the composition comprises a stearic-stearic-oleic triglyceride content of less than 10% w/w and a stearic-oleic-oleic triglyceride content of less than 10% w/w.

59-60. (canceled)

61. The composition of claim 1, wherein greater than 40% of triglycerides in the composition have one unsaturated sidechain.

62. The composition of claim 1, wherein greater than 30% of triglycerides in the composition have two unsaturated sidechains.

63. The composition of claim 1, wherein between 10% and 15% of palmitic and/or stearic fatty acids are located at the sn-2 position of triglyceride molecules in the composition.

64. (canceled)

65. A method of producing microbial oil composition according to claim 1, the method comprising the steps of:
a) providing an oleaginous yeast and a carbon source, and
b) culturing said oleaginous yeast,
thereby producing said microbial oil composition.
